SEQUENCE LISTING (36 sequences)

SEQ ID NO: 1 - DNA, 1263 bp, Homo sapiens
atgactcagattcaaggaacttgtaactttatgcaagagtctgttcttgactttgacaaaccttcaagtgcaattccaacgtcacaaccgccttcagctactccaggtagccccgtagcatctaaagaacaaaatctgtccagtcaaagtgattttcttcaagagccgttacaggtatttaacgttaatgcacctctgcctccacgaaaagaacaagaaataaaagaatccccttattcacctggctacaatcaaagttttaccacagcaagtacacaaacaccaccccagtgccaactgccatctatacatgtagaacaaactgtccattctcaagagactgcagcaaattatcatcctgatggaactattcaagtaagcaatggtagccttgccttttacccagcacagacgaatgtgtttcccagacctactcagccatttgtcaatagccggggatctgttagaggatgtactcgtggtgggagattaataaccaattcctatcggtcccctggtggttataaaggttttgatacttatagaggactcccttcaatttccaatggaaattatagccagctgcagttccaagctagagagtattctggagcaccttattcccaaagggataatttccagcagtgttataagcgaggagggacatctggtggtccacgagcaaattcgagagcagggtggagtgattcttctcaggtgagcagcccagaaagagacaacgaaacctttaacagtggtgactctggacaaggagactcccgtagcatgacccctgtggatgtgccagtgacaaatccagcagccaccatactgccagtacacgtctaccctctgcctcagcagatgcgagttgccttctcagcagccagaacctctaatctggcccctggaactttagaccaacctattgtgtttgatcttcttctgaacaacttaggagaaacttttgatcttcagcttggtagatttaattgcccagtgaatggcacttacgttttcatttttcacatgctaaagctggcagtgaatgtgccactgtatgtcaacctcatgaagaatgaagaggtcttggtatcagcctatgccaatgatggtgctccagaccatgaaactgctagcaatcatgcaattcttcagctcttccagggagaccagatatggttacgtctgcacaggggagcaatttatggaagtagctggaaatattctacgttttcaggctatcttctttatcaagattga

SEQ ID NO: 2 - protein, 420 aa, Homo sapiens
MTQIQGTCNFMQESVLDFDKPSSAIPTSQPPSATPGSPVASKEQNLSSQSDFLQEPLQVFNVNAPLPPRKEQEIKESPYSPGYNQSFTTASTQTPPQCQLPSIHVEQTVHSQETAANYHPDGTIQVSNGSLAFYPAQTNVFPRPTQPFVNSRGSVRGCTRGGRLITNSYRSPGGYKGFDTYRGLPSISNGNYSQLQFQAREYSGAPYSQRDNFQQCYKRGGTSGGPRANSRAGWSDSSQVSSPERDNETFNSGDSGQGDSRSMTPVDVPVTNPAATILPVHVYPLPQQMRVAFSAARTSNLAPGTLDQPIVFDLLLNNLGETFDLQLGRFNCPVNGTYVFIFHMLKLAVNVPLYVNLMKNEEVLVSAYANDGAPDHETASNHAILQLFQGDQIWLRLHRGAIYGSSWKYSTFSGYLLYQD

SEQ ID NO: 3 - DNA, 1230 bp, Mus musculus
atggtggtctccccctgcaagccaaagccagcttctgcacttgcttctccaaatcctcccctgtcaaagagcttccagttacctcctgcaagtgggagttcagaagccattagcacagcaccttttcaggccatgcagacagtgtttaatgttaatgcacctctgcctccacggaaagaacaagaaatgaaagaacctccttattcatctggctacaatcaaaattttacttcatcaagtacacagacagtatcccaatgccagctcccagctgtacacatagaccagacaactcagcctccagagactggtgcaggttatcatcctgatggaactgttcaagtaagcaatggcagccttgccttttacccagcacccacgagtatgtttcccagacctgctcagccatttatcagtagtcgggggactctgagagggtgttcacatggagggaggttactaatgagttcctatcagtctcctggtggctacaaaggttttgatagttacagaggacttccttcagtttcaagtgggaattacagccagctgcagctgcaagctagagaatattctggaacagcttactctcaaagggataatttccagcagtgttataaaagatcagggacatctagtggtcttcaggcaaattcaagagcagggtggagtgactcctctcaggtgagcagcccagaaagagacagcgagacttttaacagtggagactctgggctaggagactctcggagcatgaccccagtggatgtgccagtgacaagcccagcagccgccatcctgccagtccatatctatcccctgcctcagcaaatgcgagttgccttctcagctgccagaacatccaatctggctcctggaactttagaccaacctattgtgtttgatcttctcctgaacaacttgggagagacctttaatcttcagcttggtagattcaattgcccagtgaatggcacttacgtgttcatttttcacatgctaaagctggctgtgaatgtaccactgtatgtcaacctgatgaagaatgaggaggtcttggtgtcagcctatgccaacgatggtgctccagaccatgagacagcaagcaaccatgccgttctccagctcctccagggagaccagatatggctgcgcttacacaggggagcgatttatggaagtagctggaaatactctacattttcaggctatcttctttatcaagattaa

SEQ ID NO: 4 - protein, 409 aa, Mus musculus
MVVSPCKPKPASALASPNPPLSKSFQLPPASGSSEAISTAPFQAMQTVFNVNAPLPPRKEQEMKEPPYSSGYNQNFTSSSTQTVSQCQLPAVHIDQTTQPPETGAGYHPDGTVQVSNGSLAFYPAPTSMFPRPAQPFISSRGTLRGCSHGGRLLMSSYQSPGGYKGFDSYRGLPSVSSGNYSQLQLQAREYSGTAYSQRDNFQQCYKRSGTSSGLQANSRAGWSDSSQVSSPERDSETFNSGDSGLGDSRSMTPVDVPVTSPAAAILPVHIYPLPQQMRVAFSAARTSNLAPGTLDQPIVFDLLLNNLGETFNLQLGRFNCPVNGTYVFIFHMLKLAVNVPLYVNLMKNEEVLVSAYANDGAPDHETASNHAVLQLLQGDQIWLRLHRGAIYGSSWKYSTFSGYLLYQD

SEQ ID NO: 5 - DNA, 993 bp, Homo sapiens
atgggaaaactatgcctggggccgacgctctgcccggctgctgccgctgaggaaagccgggacgcggagccccgccgagagcttctttgctccggacgcccctggacgtggcgggcagccgcgagggtaaccaccatgatcccctgggtgctcctggcctgtgccctcccctgtgctgctgacccactgcttggcgcctttgctcgcagggacttccggaaaggctcccctcaactggtctgcagcctgcctggcccccagggcccacccggccccccaggagccccagggccctcaggaatgatgggacgaatgggctttcctggcaaagacggccaagatggacacgacggcgaccggggggacagcggagaggaaggtccacctggccggacaggtaaccggggaaagccaggaccaaagggcaaagccggggccattgggcgggctggcccccgtggccccaagggggtcaacggtacccccgggaagcatggcacaccaggcaagaaggggcccaagggcaagaagggggagccaggcctcccaggcccctgcagctgtggcagtggccataccaagtcagctttctcggtggcagtgaccaagagctacccacgggagcggctgcccatcaagtttgacaagattctgatgaacgagggtggccactacaatgcttccagcggcaagttcgtctgcggcgtgcctgggatctactacttcacctacgacatcacgctggccaacaagcacctggccatcggcctggtgcacaacggccagtaccgcatccggacctttgatgccaacaccggcaaccacgatgtggcctcaggctccaccatcctggctctcaagcagggtgacgaagtttggctgcagatcttctactcagagcagaacgggctcttctatgacccttactggacagacagcctctttacgggcttcctaatctatgccgaccaggatgaccccaacgaggtatag

SEQ ID NO: 6 - protein, 330 aa, Homo sapiens
MGKLCLGPTLCPAAAAEESRDAEPRRELLCSGRPWTWRAAARVTTMIPWVLLACALPCAADPLLGAFARRDFRKGSPQLVCSLPGPQGPPGPPGAPGPSGMMGRMGFPGKDGQDGHDGDRGDSGEEGPPGRTGNRGKPGPKGKAGAIGRAGPRGPKGVNGTPGKHGTPGKKGPKGKKGEPGLPGPCSCGSGHTKSAFSVAVTKSYPRERLPIKFDKILMNEGGHYNASSGKFVCGVPGIYYFTYDITLANKHLAIGLVHNGQYRIRTFDANTGNHDVASGSTILALKQGDEVWLQIFYSEQNGLFYDPYWTDSLFTGFLIYADQDDPNEV

SEQ ID NO: 7 - DNA, 885 bp, Mus musculus
atgacaatttttaaaaaggtgacaaccatgatctcctgggtactcttggcctgtgcccttccgtgtgctgctgacccaatgcttggtgcctttgctcgcagggacttccagaaggggggtcctcaactagtttgcagcctgcctggtccccaaggcccacctggccctccaggagcaccaggatcctcaggagtggtgggaagaatgggtttccctgggaaagacggccaagatggccaggacggagaccggggggacagtggagaagaaggtccacctggcaggacaggcaaccgtggaaaacaaggaccaaagggcaaagctggggccattggcagagctggccctcgaggacccaagggggtcagtggtacccccgggaagcatggcacaccgggcaagaagggacctaagggcaaaaaaggggagcctggcctcccaggcccctgcagctgcggcagtagccgagccaagtcggccttttccgtggcggtaaccaagagctacccacgcgagagactgcctatcaagtttgacaagattctgatgaacgagggtggccactacaacgcgtccagtggcaagttcgtctgcagcgtgccggggatctattactttacctatgacattacgctggccaacaaacacctggccatcggcctggtgcacaatggtcagtaccgcattcggacttttgatgccaacacgggcaaccacgacgtggcctcgggctccaccatcctagctctcaaggagggtgatgaagtctggctgcagatcttctactcagagcagaatggcctcttctacgacccttactggaccgacagcctgttcaccggcttcctcatctacgctgaccaaggagaccccaacgaggtatag

SEQ ID NO: 8 - protein, 294 aa, Mus musculus
MTIFKKVTTMISWVLLACALPCAADPMLGAFARRDFQKGGPQLVCSLPGPQGPPGPPGAPGSSGVVGRMGFPGKDGQDGQDGDRGDSGEEGPPGRTGNRGKQGPKGKAGAIGRAGPRGPKGVSGTPGKHGTPGKKGPKGKKGEPGLPGPCSCGSSRAKSAFSVAVTKSYPRERLPIKFDKILMNEGGHYNASSGKFVCSVPGIYYFTYDITLANKHLAIGLVHNGQYRIRTFDANTGNHDVASGSTILALKEGDEVWLQIFYSEQNGLFYDPYWTDSLFTGFLIYADQGDPNEV

SEQ ID NO: 9 - DNA, 3162 bp, Homo sapiens
atgtggcagcccagacggccctggccccgcgtgccctggcgctgggcgctggcgctgctggccctggttggcgcggggctgtgccacgccggcccgcagcccgggtatcccgcgcggcccagcgccaggaacaagaactggtgcgcctacatcgtgaacaagaatgtgagctgctccgtgctggagggaagtgagagttttattcaggctcagtacaactgtgcctggaaccagatgccctgtccgtcggcgctggtgtatcgagtgaacttcagacctagatatgtcactaggtataagacagtgacacagttggaatggaggtgctgtcctggctttagagggggagattgccaagaaggtcccaaagaccccgtgaagaccctccgccccacgccggctcggcctcgaaayagcttgaagaaagccacagataatgaacccagccaattctcagagcccaggaagactttgtccccaactggtacagcacaaccaagctggggggtagatccaaaagaggggcctcaggaacttcaggaaaagaagatacaggtgctagaggagaaggttcttcgactcacaaggacggttcttgacctccagtcttcccttgctggagtgagtgaaaatctcaaacatgccactcaggatgatgccagtagaacacgggcaccagggctcagcagccagcaccccaagcctgacaccactgttagtggagacacagaaacgggccagagtcctggtgtcttcaacactaaggaatctggcatgaaggacatcaagtctgaattggctgaagtcaaagatactctaaagaacaaaagtgacaagctggaagagctggatggaaaagtgaagggctacgaagggcagctcagacagctccaggaagcagctcagggcccgacggtgaccatgacaaccaacgaactctaccaagcctatgtggacagtaagatcgacgccctgagagaggagctcatggagggcatggacagaaagctggctgacctgaaaaactcatgtgagtacaagctcactggcctccagcagcagtgtgatgactatgggagcagctacctgggagtgatagagctcataggggagaaggaaacaagcctgagaaaagaaataaataacctccgagcccggctacaggagccttcagcccaggcaaattgctgcgacagtgaaaagaatggtgacattggtcaacagatcaagacattggaccagaaaatcgagagagttgctgaagccaccagaatgctgaatggaagactggacaatgagtttgaccgccttatagttccagagccagatgtggattttgatgcaaaatggaatgaactcgatgcaaggatcaatgtgacggagaagaacgctgaagaacattgcttttacattgaggaaacccttcggggcgccattaatggagaggtgggtgacttgaagcagcttgttgatcagaaaatacagtctctggaagaccgtctggggagcgttctcctacagatgaccaataacactggtgcagagctcagtcccccaggggcagcagccctgccaggagtgtcagggtcaggagatgaacgggtcatgatggaattaaaccacctgaaggacggagttcaagttgttgaagacatttgcctgctgaacatccagggaaagcctcatgggatggaaggtgccttgccaaacagggaagaccgcgcagtacgcgacagcctgcaccttttgaaatctctcaacgacacgatgcacaggaagtttcaagaaaccgaacaaaccatccagaaacttcaacaggattttagttttctttattctcaattaaaccacacagaaaatgatgtgactcatcttcaaaaggaaatgagcaattgtagagcaggtgaaaacgctggcatgggtaggttcactaaggtgggtgagcaagaaaggacagtggacaccctgccgtccccccagcaccccgtggctcattgctgcagtcagctggaggagaggtggcagaggttgcagagccaggtcatctcggagctggatgcttgtaaggaatgcacgcagggggtccagagggaggtctccatggtggagggcagggtgtctcatatggagaaaacttgcagcaagctggactctatctcaggaaatcttcagaggatcaaggaggggctcaacaagcatgtcagcagcctgtggaactgtgtcaggcagatgaacggaacgctcaggtcgcattccagagacatttctggcctgaagaattcagtccagcagttctacagccacgtcttccagatttctactgatttgcaagatctggtcaaatttcagccatcagcaaaggcgccctcgcccccgccgcccgcagaggccccgaaggagccgctgcagcccgagcccgccccgccgaggcccagcggccccgcaaccgcagaggaccctgggcgacggcccgtcctgccccagcggccccccgaggagaggccgccccagccgccaggctccaccggggtcatcgcggagacgggccaggccgggccccccgcaggcgcaggcgtgtctgggcggggtctgccgcggggcgtggacggccagaccgggagcggcaccgtccccggcgcagaaggcttcgcgggcgcaccaggatacccgaagtcacctcctgtagcttccccaggagctccggtgccttctctggtgtctttttctgcggggctcacccagaagcctttccccagtgatgggggcgttgtcctctttaacaaagtgctggtgaacgacggggatgtttacaaccccagcaccggggtcttcacggctccttatgatgggcgctacctgatcacggccaccctcacccccgagagagacgcctacgtggaagcagtgctgtcggtctccaacgccagcgtggcccagctgcataccgctgggtacaggagagagttcctggaataccaccgccctccaggagctttgcatacctgcgggggcccgggggcattccacctcatcgtgcacctgaaggcgggagatgcagtcaacgtcgtggtgactgggggcaagctggctcacacagactttgatgaaatgtactccacatttagtggggttttcttatatcctttcctttcccacctctaa

SEQ ID NO: 10 - protein, 1053 aa, Homo sapiens
MWQPRRPWPRVPWRWALALLALVGAGLCHAGPQPGYPARPSARNKNWCAYIVNKNVSCSVLEGSESFIQAQYNCAWNQMPCPSALVYRVNFRPRYVTRYKTVTQLEWRCCPGFRGGDCQEGPKDPVKTLRPTPARPRNSLKKATDNEPSQFSEPRKTLSPTGTAQPSWGVDPKEGPQELQEKKIQVLEEKVLRLTRTVLDLQSSLAGVSENLKHATQDDASRTRAPGLSSQHPKPDTTVSGDTETGQSPGVFNTKESGMKDIKSELAEVKDTLKNKSDKLEELDGKVKGYEGQLRQLQEAAQGPTVTMTTNELYQAYVDSKIDALREELMEGMDRKLADLKNSCEYKLTGLQQQCDDYGSSYLGVIELIGEKETSLRKEINNLRARLQEPSAQANCCDSEKNGDIGQQIKTLDQKIERVAEATRMLNGRLDNEFDRLIVPEPDVDFDAKWNELDARINVTEKNAEEHCFYIEETLRGAINGEVGDLKQLVDQKIQSLEDRLGSVLLQMTNNTGAELSPPGAAALPGVSGSGDERVMMELNHLKDGVQVVEDICLLNIQGKPHGMEGALPNREDRAVRDSLHLLKSLNDTMHRKFQETEQTIQKLQQDFSFLYSQLNHTENDVTHLQKEMSNCRAGENAGMGRFTKVGEQERTVDTLPSPQHPVAHCCSQLEERWQRLQSQVISELDACKECTQGVQREVSMVEGRVSHMEKTCSKLDSISGNLQRIKEGLNKHVSSLWNCVRQMNGTLRSHSRDISGLKNSVQQFYSHVFQISTDLQDLVKFQPSAKAPSPPPPAEAPKEPLQPEPAPPRPSGPATAEDPGRRPVLPQRPPEERPPQPPGSTGVIAETGQAGPPAGAGVSGRGLPRGVDGQTGSGTVPGAEGFAGAPGYPKSPPVASPGAPVPSLVSFSAGLTQKPFPSDGGVVLFNKVLVNDGDVYNPSTGVFTAPYDGRYLITATLTPERDAYVEAVLSVSNASVAQLHTAGYRREFLEYHRPPGALHTCGGPGAFHLIVHLKAGDAVNVVVTGGKLAHTDFDEMYSTFSGVFLYPFLSHL

SEQ ID NO: 11 - DNA, 444 bp, Mus musculus
atgcagtttgtgtgtctcacacatttgtccagtttctctgtactagcccttggtctgccaaggggagtctctggactttcagaagataggcaggtccacatgctctggcttctctctgccctaggggtgccgctgcccactctggtgtccttttcagccggtcttacccagaagccttttcccagcgatggaggcgttgttctcttcaataaggtgctggtgaacgatggggatgtatacaaccccaacactgggatcttcaccgcaccatacgacgggcgctacttgatcacagccaccctcacccctgagagagacacctatgtggaagccgtcctgtctgtctccaacgccagtgttgcccagctgcacacggctgggtacaggagagagttcctggaataccatcgtcccccttgggctgtgcatacctgtgggggcccg

SEQ ID NO: 12 - protein, 148 aa, Mus musculus
MQFVCLTHLSSFSVLALGLPRGVSGLSEDRQVHMLWLLSALGVPLPTLVSFSAGLTQKPFPSDGGVVLFNKVLVNDGDVYNPNTGIFTAPYDGRYLITATLTPERDTYVEAVLSVSNASVAQLHTAGYRREFLEYHRPPWAVHTCGGP

SEQ ID NO: 13 - DNA, 990 bp, Homo sapiens
atgctgccgcttctgctgggcctgctgggcccagcggcctgctgggccctgggcccgacccccggcccgggatcctctgagctgcgctcggccttctcggcggcacgcaccacccccctggagggcacgtcggagatggcggtgaccttcgacaaggtgtacgtgaacatcgggggcgacttcgatgtggccaccggccagtttcgctgccgcgtgcccggcgcctacttcttctccttcacggctggcaaggccccgcacaagagcctgtcggtgatgctggtgcgaaaccgcgacgaggtgcaggcgctggccttcgacgagcagcggcggccaagcgcgcggcgcgcagccagccagagcgccatgctgcagctcgactacggcgacacagtgtggctgcggctgcatggcgccccgcactacgcgctaggcgcgcccggcgccaccttcagcggctacctagtctacgccgacgccgacgctgacgcgcctgcgcgcgggccgcccgcgccccccgagccgcgctcggccttctcggcggcgcgcacgcgcagcttggtgggctcggacgctggccccgggccgcggcaccaaccactcgccttcgacaccgagttcgtcaacattggcggcgacttcgacgcggcggccggcgtgttccgctgccgtctgcccggcgcctacttcttctccttcacgctgggcaagctgccgcgtaagacgctgtcggttaagctgatgaagaaccgcgacgaggtgcaggccatgatttacgacgacggcgcgtcgcggcgccgcgagatgcagagccagagcgtgatgctggccctgcggcgcggcgacgccgtctggctgctcagccacgaccacgacggctacggcgcctacagcaaccacggcaagtacatcaccttctccggcttcctggtgtaccccgacctcgcccccgccgccccgccgggcctcggggcctcggagctactgtga

SEQ ID NO: 14 - protein, 329 aa, Homo sapiens
MLPLLLGLLGPAACWALGPTPGPGSSELRSAFSAARTTPLEGTSEMAVTFDKVYVNIGGDFDVATGQFRCRVPGAYFFSFTAGKAPHKSLSVMLVRNRDEVQALAFDEQRRPSARRAASQSAMLQLDYGDTVWLRLHGAPHYALGAPGATFSGYLVYADADADAPARGPPAPPEPRSAFSAARTRSLVGSDAGPGPRHQPLAFDTEFVNIGGDFDAAAGVFRCRLPGAYFFSFTLGKLPRKTLSVKLMKNRDEVQAMIYDDGASRRREMQSQSVMLALRRGDAVWLLSHDHDGYGAYSNHGKYITFSGFLVYPDLAPAAPPGLGASELL

SEQ ID NO: 15 - DNA, 981 bp, Mus musculus
atgctgctgctcttgctgggcttcctaggcccggcggcctgctgggcactgggcccggctggccctggctcctcggagctgcggtcagccttctcggcggctcgcaccaccccgctggagggcacgtcggagatggcggtgaccttcgacaaggtgtacgtgaacatcgggggtgacttcgacgcagccaccgggcggttccgctgtcgcgtgccgggcgcctacttcttctccttcacggccggcaaggccccgcacaagagcctgtcggtgatgctggtgcgcaaccgcgacgaggtgcaggcgctggctttcgacgagcagcgacggccaggcgcgcggcgcgcggccagccagagcgccatgctgcagctcgactacggcgacacggtgtggctgcggctgcacggcgctccgcagtacgcgctcggcgcgccgggcgccaccttcagcggctacctggtgtacgcggacgccgacgccgacgcgcctgcgcgcggccccgcggccccggagccgcgctcggccttctccgcggcgcgcacgcgcagcctggtgggctcggacgccgcccccggcccgcgccaccggccgttggccttcgacaccgagctggtaaacataggtggcgacttcgacgcggcggccggcgtgttccgctgccgcctgccgggagcctatttcttctccttcacgctgggcaagctgccgcgcaagacgctgtcggtgaagctgatgaagaaccgcgacgaggtgcaggccatgatttacgacgacggcgcttcgaggcgccgtgagatgcagagtcagagcgtgatgctgccgctgcggcgcggcgacgccgtctggctacttagccacgatcacgatggctatggcgcctacagcaaccacggcaagtacatcactttctcaggcttcctggtgtaccctgacctcgccgccgccggcccgccggccctcaagcccccagagctctga

SEQ ID NO: 16 - protein, 326 aa, Mus musculus
MLLLLLGFLGPAACWALGPAGPGSSELRSAFSAARTTPLEGTSEMAVTFDKVYVNIGGDFDAATGRFRCRVPGAYFFSFTAGKAPHKSLSVMLVRNRDEVQALAFDEQRRPGARRAASQSAMLQLDYGDTVWLRLHGAPQYALGAPGATFSGYLVYADADADAPARGPAAPEPRSAFSAARTRSLVGSDAAPGPRHRPLAFDTELVNIGGDFDAAAGVFRCRLPGAYFFSFTLGKLPRKTLSVKLMKNRDEVQAMIYDDGASRRREMQSQSVMLPLRRGDAVWLLSHDHDGYGAYSNHGKYITFSGFLVYPDLAAAGPPALKPPEL

SEQ ID NO: 17 - DNA, 732 bp, Homo sapiens
atgaggccactcctcgtcctgctgctcctgggcctggcggccggctcgcccccactggacgacaacaagatccccagcctctgcccggggcaccccggccttccaggcacgccgggccaccatggcagccagggcttgccgggccgcgatggccgcgacggccgcgacggcgcgcccggggctccgggagagaaaggcgagggcgggaggccgggactgccgggacctcgaggggaccccgggccgcgaggagaggcgggacccgcggggcccaccgggcctgccggggagtgctcggtgcctccgcgatccgccttcagcgccaagcgctccgagagccgggtgcctccgccgtctgacgcacccttgcccttcgaccgcgtgctggtgaacgagcagggacattacgacgccgtcaccggcaagttcacctgccaggtgcctggggtctactacttcgccgtccatgccaccgtctaccgggccagcctgcagtttgatctggtgaagaatggcgaatccattgcctctttcttccagtttttcggggggtggcccaagccagcctcgctctcggggggggccatggtgaggctggagcctgaggaccaagtgtgggtgcaggtgggtgtgggtgactacattggcatctatgccagcatcaagacagacagcaccttctccggatttctggtgtactccgactggcacagctccccagtctttgcttag

SEQ ID NO: 18 - protein, 243 aa, Homo sapiens
MRPLLVLLLLGLAAGSPPLDDNKIPSLCPGHPGLPGTPGHHGSQGLPGRDGRDGRDGAPGAPGEKGEGGRPGLPGPRGDPGPRGEAGPAGPTGPAGECSVPPRSAFSAKRSESRVPPPSDAPLPFDRVLVNEQGHYDAVTGKFTCQVPGVYYFAVHATVYRASLQFDLVKNGESIASFFQFFGGWPKPASLSGGAMVRLEPEDQVWVQVGVGDYIGIYASIKTDSTFSGFLVYSDWHSSPVFA

SEQ ID NO: 19 - DNA, 732 bp, Mus musculus
atgaggccacttcttgcccttctgcttctgggtctggtgtcaggctctcctcctctggacgacaacaagatccccagcctgtgtcccgggcagcccggccttccaggcacaccaggtcaccatggcagccaaggcctgcctggccgtgacggcsgtgatggccgcgacggtgcacccggagctccgggagagaaaggcgagggcgggagaccgggactacctggcccacgtggggagcccgggccgcgtggagaggcagggcccatgggggctatcgggcctgcgggsgagtgctcggtacccccacgatcagccttcagtgccaagcgatccgagagccgggtacctccgccagccgacacacccctacctttcgaccgtgtgctgctaaatgagcagggccatttcgaccccactactggcaagttcacctgccaagtgcctggcgtctactactttgctgtgcacgccactgtctaccgggccagcttgcagtttgatcttgtcaaaaacgggcagtccatcgcctctttcttccagtattttggggggtggcccaagccagcctcgctctcagggggtgcgatggtaaggctagaacctgaggaccaggtgtgggtgcaggtgggcgtgggtgattacattggcatctatgccagcatcaagacagacagtaccttctctggatttctcgtctattctgactggcacagctccccagtcttcgcttaa

SEQ ID NO: 20 - protein, 243 aa, Mus musculus
  MISC_FEATURE (52)..(52): unsure
MRPLLALLLLGLVSGSPPLDDNKIPSLCPGQPGLPGTPGHHGSQGLPGRDGXDGRDGAPGAPGEKGEGGRPGLPGPRGEPGPRGEAGPMGAIGPAGECSVPPRSAFSAKRSESRVPPPADTPLPFDRVLLNEQGHFDPTTGKFTCQVPGVYYFAVHATVYRASLQFDLVKNGQSIASFFQYFGGWPKPASLSGGAMVRLEPEDQVWVQVGVGDYIGIYASIKTDSTFSGFLVYSDWHSSPVFA

SEQ ID NO: 21 - DNA, 2850 bp, Homo sapiens
atgatcctgagcttgctgttcagccttgggggccccctgggctgggggctgctgggggcatgggcccaggcttccagtactagcctctctgatctgcagagctccaggacacctggggtctggaaggcagaggctgaggacaccagcaaggaccccgttggacgtaactggtgcccctacccaatgtccaagctggtcaccttactagctctttgcaaaacagagaaattcctcatccactcgcagcagccgtgtccgcagggagctccagactgccagaaagtcaaagtcatgtaccgcatggcccacaagccagtgtaccaggtcaagcagaaggtgctgacctctttggcctggaggtgctgccctggctacacgggccccaactgcgagcaccacgattccatggcaatccctgagcctgcagatcctggtgacagccaccaggaacctcaggatggaccagtcagcttcaaacctggccaccttgctgcagtgatcaatgaggttgaggtgcaacaggaacagcaggaacatctgctgggagatctccagaatgatgtgcaccgggtggcagacagcctgccaggcctgtggaaagccctgcctggtaacctcacagctgcagtgatggaagcaaatcaaacagggcacgagttccctgatagatccttggagcaggtgctgctaccccacgtggacaccttcctacaagtgcatttcagccccatctggaggagctttaaccaaagcctgcacagccttacccaggccataagaaacctgtctcttgacgtggaggccaaccgccaggccatctccagagtccaggacagtgccgtggccagggctgacttccaggagcttggtgccaaatttgaggccaaggtccaggagaacactcagagagtgggtcagctgcgacaggacgtggaggaccgcctgcacgcccagcactttaccctgcaccgctcgatctcagagctccaagccgatgtggacaccaaattgaagaggctgcacaaggctcaggaggccccagggaccaatggcagtctggtgttggcaacgcctggggctggggcaaggcctgagccggacagcctgcaggccaggctgggccagctgcagaggaacctctcagagctgcacatgaccacggcccgcagggaggaggagttgcagtacaccctggaggacatgagggccaccctgacccggcacgtggatgagatcaaggaactgtactccgaatcggacgagactttcgatcagattagcaaggtggagcggcaggtggaggagctgcaggtgaaccacacggcgctccgtgagctgcgcgtgatcctgatggagaagtctctgatcatggaggagaacaaggaggaggtggagcggcagctcctggagctcaacctcacgctgcagcacctgcagggtggccatgccgacctcatcaagtacgtgaaggactgcaattgccagaagctctatttagacctggacgtcatccgggagggccagagggacgccacgcgtgccctggaggagacccaggtgagcctggacgagcggcggcagctggacggctcctccctgcaggccctgcagaacgccgtggacgccgtgtcgctggccgtggacgcgcacaaagcggagggcgagcgggcgcgggcggccacgtcgcggctccggagccaagtgcaggcgctggatgacgaggtgggcgcgctgaaggcggccgcggccgaggcccgccacgaggtgcgccagctgcacagcgccttcgccgccctgctggaggacgcgctgcggcacgaggcggtgctggccgcgctcttcggggaggaggtgctggaggagatgtctgagcagacgccgggaccgctgcccctgagctacgagcagatccgcgtggccctgcaggacgccgctagcgggctgcaggagcaggcgctcggctgggacgagctggccgcccgagtgacggccctggagcaggcctcggagcccccgcggccggcagagcacctggagcccagccacgacgcgggccgcgaggaggccgccaccaccgccctggccgggctggcgcgggagctccagagcctgagcaacgacgtcaagaatgtcgggcggtgctgcgaggccgaggccggggccggggccgcctccctcaacgcctcccttgacggcctccacaacgcactcttcgccactcagcgcagcttggagcagcaccagcggctcttccacagcctctttgggaacttccaagggctcatggaagccaacgtcagcctggacctggggaagctgcagaccatgctgagcaggaaagggaagaagcagcagaaagacctggaagctccccggaagagggacaagaaggaagcggagcctttggtggacatacgggtcacagggcctgtgccaggtgccttgggcgcggcgctctgggaggcaggatcccctgtggccttctatgccagcttttcagaagggacgsctgccctgcagacagtgaagttcaacaccacatacatcaacattggcagcagctacttccctgaacatggctacttccgagcccctgagcgtggtgtctacctgtttgcagtgagcgttgaatttggcccagggccaggcaccgggcagctggtgtttggaggtcaccatcggactccagtctgtaccactgggcaggggagtggaagcacagcaacggtctttgccatggctgagctgcagaagggtgagcgagtatggtttgagttaacccagggatcaataacaaagagaagcctgtcgggcactgcatttgggggcttcctgatgtttaagacctga

SEQ ID NO: 22 - protein, 949 aa, Homo sapiens
MILSLLFSLGGPLGWGLLGAWAQASSTSLSDLQSSRTPGVWKAEAEDTSKDPVGRNWCPYPMSKLVTLLALCKTEKFLIHSQQPCPQGAPDCQKVKVMYRMAHKPVYQVKQKVLTSLAWRCCPGYTGPNCEHHDSMAIPEPADPGDSHQEPQDGPVSFKPGHLAAVINEVEVQQEQQEHLLGDLQNDVHRVADSLPGLWKALPGNLTAAVMEANQTGHEFPDRSLEQVLLPHVDTFLQVHFSPIWRSFNQSLHSLTQAIRNLSLDVEANRQAISRVQDSAVARADFQELGAKFEAKVQENTQRVGQLRQDVEDRLHAQHFTLHRSISELQADVDTKLKRLHKAQEAPGTNGSLVLATPGAGARPEPDSLQARLGQLQRNLSELHMTTARREEELQYTLEDMRATLTRHVDEIKELYSESDETFDQISKVERQVEELQVNHTALRELRVILMEKSLIMEENKEEVERQLLELNLTLQHLQGGHADLIKYVKDCNCQKLYLDLDVIREGQRDATRALEETQVSLDERRQLDGSSLQALQNAVDAVSLAVDAHKAEGERARAATSRLRSQVQALDDEVGALKAAAAEARHEVRQLHSAFAALLEDALRHEAVLAALFGEEVLEEMSEQTPGPLPLSYEQIRVALQDAASGLQEQALGWDELAARVTALEQASEPPRPAEHLEPSHDAGREEAATTALAGLARELQSLSNDVKNVGRCCEAEAGAGAASLNASLDGLHNALFATQRSLEQHQRLFHSLFGNFQGLMEANVSLDLGKLQTMLSRKGKKQQKDLEAPRKRDKKEAEPLVDIRVTGPVPGALGAALWEAGSPVAFYASFSEGTAALQTVKFNTTYINIGSSYFPEHGYFRAPERGVYLFAVSVEFGPGPGTGQLVFGGHHRTPVCTTGQGSGSTATVFAMAELQKGERVWFELTQGSITKRSLSGTAFGGFLMFKT

SEQ ID NO: 23 - DNA, 1974 bp, Mus musculus
  misc_feature (1288)..(1290): unsure
atgatcccgacactgctgctgggctttggggtgtacctgagctggggactgctagggtcctgggcacaggaccccggtaccaagttctcccatctcaataggcccggcatgcctgaaggctggagactaggggctgaggataccagcagagaccccatcagacggaactggtgtccttaccagaagtccaggctggtcacctttgtagctgcttgcaaaacagagaaattcctggtccattcacagcagccatgtccacagggagcccctgactgccagggagtcagagtcatgtatcgagtggcccagaagccagtgtaccaggtccagcagaaggtgctgatctctgtggactggcggtgctgcccagggttccagggaccagactgccaggaccacaatcccacagcaaaccctgagcccacagagccaagtggtaaactccaggagacttgggactcgatggatggctttgaacttggtcaccctgtcccagagtttaatgagattaaggtgccacaggaacaacaggaaaacctgcttcaaaatctccagaatgatgcccagtcagtagaagatggctttccaggctcttgggaagccccacccagcaacctcacagatgagatgacagaagccaatctaacagaattcgagtttcctggcaggacatcagagcacctgctgcagccccatattgatgcattcctgaaagcacacttcagtcccatctggaagaacttcaacgacagcttgcacagcctctcccaggccatcagaaacttgtctcttgatgtggaggccaatcaccaggccatcaagatgatccaggagggcacagtggctagggctgacttccaagagcttggtgccaagtttgaggccaaggtccagcagaatagccagagactgggccaactgtggcaggatgtggaggaccagctgcatgcccagcgccgatcggtgcatcatgccctctctgaggtccaggctgaggtgagcaccaagttaaagcagcttgtcaaggctcaggaacttccaggggccaatggcagcctggtggtggtatctgcagcagcggcagcaaggccagagccagagagcctgcaggccaggctagggcagctgcagagaaacctctctgctctgcacatggtcactagccagagggaggaggagttgcagagcaccctcaagaacatggacagcgtcctgaagcagcacgccgaagagatcaaggagctctattctgaatcggatgagaccttcgaccagatcagcaaggtannncagtgctgtgaggcctcntgggctgcctcaatcaatagctcccttgaagacctacacagcatgctcttcgacacccagcacggcttgagacagcaccggcagctcttccacaacctcttccagaacttccaaggtctggtggcaagcaacatcagcctagacttggggaagctgcaggccatgttgagtaagaaagataagaagcaaccgagaggcccaggagaatcccggaagagggataagaagcaagtggtgatgtctacagacgcacacgccaaaggtctggagctctgggagacaggctcccctgtggccttctatgccggttcttcagaaggggccactgctctgcagatggtgaagttcaacaccacatccatcaatgtgggcagcagctactttcctgaacatggctacttccgagctcccaaacgtggcatctacttatttgctgtgagcattacatttggcccaggcccaggaatggggcagctggtatttgaaggtcatcaccgggttccagtctacagtacggaacagaggggtgggagcacagccaccacttttgctatggtagagctgcaaaagggtgagagagcgtggtttgagttagtccaagggtcagcaaccaaagggagccgaccaggcactgcatttgggggcttcctgatgttcaagacctga

SEQ ID NO: 24 - protein, 657 aa, Mus musculus
  MISC_FEATURE (430)..(430): unsure
MIPTLLLGFGVYLSWGLLGSWAQDPGTKFSHLNRPGMPEGWRLGAEDTSRDPIRRNWCPYQKSRLVTFVAACKTEKFLVHSQQPCPQGAPDCQGVRVMYRVAQKPVYQVQQKVLISVDWRCCPGFQGPDCQDHNPTANPEPTEPSGKLQETWDSMDGFELGHPVPEFNEIKVPQEQQENLLQNLQNDAQSVEDGFPGSWEAPPSNLTDEMTEANLTEFEFPGRTSEHLLQPHIDAFLKAHFSPIWKNFNDSLHSLSQAIRNLSLDVEANHQAIKMIQEGTVARADFQELGAKFEAKVQQNSQRLGQLWQDVEDQLHAQRRSVHHALSEVQAEVSTKLKQLVKAQELPGANGSLVVVSAAAAARPEPESLQARLGQLQRNLSALHMVTSQREEELQSTLKNMDSVLKQHAEEIKELYSESDETFDQISKVXQCCEASWAASINSSLEDLHSMLFDTQHGLRQHRQLFHNLFQNFQGLVASNISLDLGKLQAMLSKKDKKQPRGPGESRKRDKKQVVMSTDAHAKGLELWETGSPVAFYAGSSEGATALQMVKFNTTSINVGSSYFPEHGYFRAPKRGIYLFAVSITFGPGPGMGQLVFEGHHRVPVYSTEQRGGSTATTFAMVELQKGERAWFELVQGSATKGSRPGTAFGGFLMFKT

SEQ ID NO: 25 - DNA, 2763 bp, Homo sapiens
atgtaccgccgcttcctccgccctcgctaccgtgtggcctacaagacagtgaccgacatggagtggaggtgctgtcagggttatgggggcgatgactgtgctgagagtcccgctccagcgctggggcctgcgtcttccacaccacggcccctggcccggcctgcccgccccaacctctctggctccagtgcaggcagccccctcagtggactggggggagaaggtcctggggagtcagagaaggtgcagcagctggaggaacaggtgcagagcctgaccaaggagctgcaaggcctgcggggcgtcctgcaaggactgagcgggcgcctggcagaggatgtgcagagggctgtggagacggccttcaacgggaggcagcagccagctgacgcggctgcccgccctggggtgcatgaaaccctcaatgagatccagcaccagctgcagctcctggacacccgcgtctccacccacgaccaggagctgggtcacctcaacaaccatcatggcggcagcagcagcagtgggggcagcagggccccagccccagcctcagcccctccgggccccagtgaggagctgctgcggcagctggagcagcggttgcaggagtcctgctccgtgtgcctggccgggctagatggcttccgccggcagcagcaggaggacagggagcggctgcgagcgatggagaagctgctggcctcggtggaggagcggcaacggcacctcgcagggctggcggtgggccgcaggccccctcaggaatgctgctctccagagctgggccggcgactggcagagctggagcgcaggctggatgtcgtggccggctcagtgacagtgctgagtgggcggcgaggcacagagctgggaggagccgcggggcagggaggccaccccccaggctacaccagcttggcctcccgcctgtctcgcctggaggaccgcttcaactccaccctgggcccttcggaggagcaggaggagagctggcctggggctcctggggggctgagccactggctgcctgctgcccggggccgactagagcagttgggggggctgctggccaatgtgagcggggagctgggggggcggttggatctgttggaggagcaggtggcaggggccatgcaggcatgcgggcagctctgctctggggcccctggggagcaggactctcaagtcagcgagatcctcagtgccttggagcgcagggtgctggacagtgaggggcagctgcggctggtgggctccggcctgcacacggtggaagcagcgggggaggcccggcaggccacgctggagggattacaagaggttgtgggccggctccaggatcgtgtggatgcccaggatgagacagctgcagagttcacactacggctgaatctcactgcggcccggctaggccaactggaggggctgctgcaggcccatggggatgagggctgtggggcctgtggcggagtccaagaggaactaggccgccttcgggatggtgtggagcgctgctcctgccccctgttgcctcctcggggtcctggggctggtccaggtgttgggggcccaagccgtgggcccctggacggcttcagcgtgtttgggggcagctcaggctcagccctgcaggccctgcaaggagagctctctgaggttattctcagcttcagctccctcaatgactcactgaatgagctccagaccactgtggagggccagggcgctgatctggctgacctgggggcaaccaaggaccgtatcatttctgagattaacaggctgcagcaggaggccacagagcatgctacagagagtgaagagcgcttccgaggcctagaggagggacaagcacaggccggccagtgccccagcttagaggggcgattgggccgtcttgagggtgtctgtgaacggttggacactgtggctgggggactgcagggcctgcgcgagggcctttccagacacgtggctgggctctgggctgggctccgggaaaccaacaccaccagccagatgcaggcagccctgctggagaagctggtcgggggacaggcgggcctgggcaggcggctgggtgcccttaacagctccctgcagctcctggaggaccgtctgcaccagctcagcctgaaggacctcactgggcctgcaggagaggctgggcccccagggcctcctgggctgcagggacccccaggccctgctggacctccaggatcaccaggcaaggacgggcaagagggccccatcgggccaccaggtcctcaaggtgaacagggagtggagggggcaccagcagcccctgtgccccaagtggcattttcagctgctctgagtttgccccggtctgaaccaggcacggtccccttcgacagagtcctgctcaatgatggaggctattatgatccagagacaggcgtgttcacagcgccactggctggacgctacttgctgagcgcggtgctgactgggcaccggcacgagaaagtggaggccgtgctgtcccgctccaaccagggcgtggcccgcgtagactccggtggctacgagcctgagggcctggagaataagccggtggccgagagccagcccagcccgggcaccctgggcgtcttcagcctcatcctgccgctgcaggccggggacacggtctgcgtcgacctggtcatggggcagctggcgcactcggaggagccgctcaccatcttcagcggggccctgctctatggggacccagagcttgaacacgcgtag

SEQ ID NO: 26 - protein, 920 aa, Homo sapiens
MYRRFLRPRYRVAYKTVTDMEWRCCQGYGGDDCAESPAPALGPASSTPRPLARPARPNLSGSSAGSPLSGLGGEGPGESEKVQQLEEQVQSLTKELQGLRGVLQGLSGRLAEDVQRAVETAFNGRQQPADAAARPGVHETLNEIQHQLQLLDTRVSTHDQELGHLNNHHGGSSSSGGSRAPAPASAPPGPSEELLRQLEQRLQESCSVCLAGLDGFRRQQQEDRERLRAMEKLLASVEERQRHLAGLAVGRRPPQECCSPELGRRLAELERRLDVVAGSVTVLSGRRGTELGGAAGQGGHPPGYTSLASRLSRLEDRFNSTLGPSEEQEESWPGAPGGLSHWLPAARGRLEQLGGLLANVSGELGGRLDLLEEQVAGAMQACGQLCSGAPGEQDSQVSEILSALERRVLDSEGQLRLVGSGLHTVEAAGEARQATLEGLQEVVGRLQDRVDAQDETAAEFTLRLNLTAARLGQLEGLLQAHGDEGCGACGGVQEELGRLRDGVERCSCPLLPPRGPGAGPGVGGPSRGPLDGFSVFGGSSGSALQALQGELSEVILSFSSLNDSLNELQTTVEGQGADLADLGATKDRIISEINRLQQEATEHATESEERFRGLEEGQAQAGQCPSLEGRLGRLEGVCERLDTVAGGLQGLREGLSRHVAGLWAGLRETNTTSQMQAALLEKLVGGQAGLGRRLGALNSSLQLLEDRLHQLSLKDLTGPAGEAGPPGPPGLQGPPGPAGPPGSPGKDGQEGPIGPPGPQGEQGVEGAPAAPVPQVAFSAALSLPRSEPGTVPFDRVLLNDGGYYDPETGVFTAPLAGRYLLSAVLTGHRHEKVEAVLSRSNQGVARVDSGGYEPEGLENKPVAESQPSPGTLGVFSLILPLQAGDTVCVDLVMGQLAHSEEPLTIFSGALLYGDPELEHA

SEQ ID NO: 27 - DNA, 2416 bp, Mus musculus
aaaagcaggctggtaccggtccggaattcccgggatatcgtcgacccacgcgtccggcccgggcgtgcatgaaaccctcagtgagatccagcagcagctgcagctcctggacaaccgtgtctccactcatgaccaggagctgggccaccttaacaaccatcataatggaggccctggtggaggtggcagggcctcaggccctgtcccagttccttctggccccagtgaagaactgttaaggcagctggaacggcagctgcaagagtcttgctcagtgtgcctgacggggctggatggcttccgccagcagcagcaagaggatagggagcggctgcgaaccctggagaagctaatgtcctctatggaggagcggcagcaacagcttgtgggacctgccatggccaggagaccccctcaggaatgctgccccccagagctgggtcgacgagtgtctgagctggagcgaaggctagatgtagtgactggctcactgacagtgctaagtggacgcagaggttctgagcttggaggagcagctgggcaggggggccaccctccaggctacaccagcttggcctcccgcctttctcgcctggaggaccgcttcaactctaccctaggtccctcagaggagcaagagaagaactggcctggaggaccagggaggctgggccactggttgcctgctgctccaggacggctagaaaagctggagggactactagccaatgtgagcagggagctgggtggccgcatggatctgctggaagagcaggtggcaggggctgttcggacttgtgggcagatttgctctggggcacccggggaacaggattctcgggtcaatgagatcctcagtgccttggaacgcagggtgctggacagcgagggccggttacagctagtgggctctggcttgcatgaagcagaggcagcaggggaggctcagcaggccgtgttggagggactgcaagggctcctgagccggcttcgggagcgcatggatgcacaggaggagactgcagcagaaatcttactgcgcctcaatcttaccgcagcccagctaagccagctggagggtctgctgcaagcccgtggggatgagggttgtggcgcctgtggtggtgtccaggaggagctgggccgccttcgggatggtgtggaacgttgctcctgcccattgttacctccacggggccctggagctggcccaggggttgggggaccaagccgtgggcctctggatggtttcagtgtgtttgggggcagttcaggctcagccctccaggcccttcaaggagaactctctgaggttattctcaccttcagctccctgaacgactcactccacgagctccagaccactgtggagggtcagggtgccgatctggctgacctgggggccaccaaggacagcatcatctctgaaatcaacagactacagcaggaggccacggagcacgtcacagagagcgaggagcgcttccgaggcctggaggagggccaggcacaggctggccaatgccctagcttagaggggcgattaggccgccttgagggagtctgcgaacgactggacaccgtggcagggggactacaaggcctacgtgaaggcctctccagacacgtggctgggctctgggctgcagtacgggaaagcaacagcaccagcctgacacaggccgccctgctggagaagctgctggggggccaggcaggcctgggcaggcggcttggtgccctcaacaattccctgctgctcctggaagaccgtctgcagcaacttagcctaaaggacttcactggaccttcaggcaaggctgggcccccagggcctcccgggctacaagggccttcaggccctgcaggacctccaggacctcctggcaaagacggacaacagggggccattggcccaccaggtcctcaaggggagcagggagcggagggagcaccagcagcccctgtgcctagggtagcattttcagctgccctgagtttgccacggtcagaacctggcacagtccccttcgacagagtcttgctcaatgatggaggctactatgacccagagacaggtgtattcactgcaccactggctggacgctatttgctgagcgctgtacttactgggcaccggcatgagaaagtggaagcagtattgtcacgctccaacctgggcgtggcccgcatagactcgggaggctatgaacccgagggactggagaataagcctgtggccgaaagtcagcccagcccaggcgctctgggcgtcttcagcctcattctgccactgcaggtcggagacactgtctgcatcgacctggtcatggggcagctggcacactccgaggagccgctcaccatcttcagcggagccctgctctacgaggacacagagcttgaacaggtgtag

SEQ ID NO: 28 - protein, 804 aa, Mus musculus
KAGWYRSGIPGISSTHASGPGVHETLSEIQQQLQLLDNRVSTHDQELGHLNNHHNGGPGGGGRASGPVPVPSGPSEELLRQLERQLQESCSVCLTGLDGFRQQQQEDRERLRTLEKLMSSMEERQQQLVGPAMARRPPQECCPPELGRRVSELERRLDVVTGSLTVLSGRRGSELGGAAGQGGHPPGYTSLASRLSRLEDRFNSTLGPSEEQEKNWPGGPGRLGHWLPAAPGRLEKLEGLLANVSRELGGRMDLLEEQVAGAVRTCGQICSGAPGEQDSRVNEILSALERRVLDSEGRLQLVGSGLHEAEAAGEAQQAVLEGLQGLLSRLRERMDAQEETAAEILLRLNLTAAQLSQLEGLLQARGDEGCGACGGVQEELGRLRDGVERCSCPLLPPRGPGAGPGVGGPSRGPLDGFSVFGGSSGSALQALQGELSEVILTFSSLNDSLHELQTTVEGQGADLADLGATKDSIISEINRLQQEATEHVTESEERFRGLEEGQAQAGQCPSLEGRLGRLEGVCERLDTVAGGLQGLREGLSRHVAGLWAAVRESNSTSLTQAALLEKLLGGQAGLGRRLGALNNSLLLLEDRLQQLSLKDFTGPSGKAGPPGPPGLQGPSGPAGPPGPPGKDGQQGAIGPPGPQGEQGAEGAPAAPVPRVAFSAALSLPRSEPGTVPFDRVLLNDGGYYDPETGVFTAPLAGRYLLSAVLTGHRHEKVEAVLSRSNLGVARIDSGGYEPEGLENKPVAESQPSPGALGVFSLILPLQVGDTVCIDLVMGQLAHSEEPLTIFSGALLYEDTELEQV

SEQ ID NO: 29 - DNA, 2235 bp, Homo sapiens
atggctgtgctgcctggccctctgcagctgctgggagtgctgcttaccatttccctgagttccatcaggctcattcaggctggtgcctactatgggatcaagccgctgccacctcaaattcctcctcagatgccaccacaaattccacaataccagcccctgggtcagcaagtacctcacatgcctttggccaaagatggccttgccatgggcaaggagatgccccacttgcagtatggcaaagagtatccacacctaccccaatatatgaaggaaattcaaccggcgccaagaatgggcaaggaagccgtacccaagaaaggcaaagaaataccattagccagtttacgaggggaacaaggtccccgtggagagcctggcccaagaggaccacctgggccccctggtttaccaggtcatgggatacctggaattaaaggaaaaccagggccacagggatatccaggagttggaaagccaggtatgcctggaatgccagggaagccaggagccatgggcatgcctggggcaaaaggagaaattggacagaaaggggaaattgggcctatggggatcccaggaccacaaggacctccagggcctcatggacttcctggcattgggaagccaggtgggccagggttaccagggcaaccaggaccaaagggtgatcgaggacccaaaggactaccaggacctcaaggccttcggggtcctaaaggagacaagggcttcgggatgccaggtgcgccaggtgtaaaggggcctccagggatgcacggccctcccggccctgttggactgccaggagtgggcaaaccaggagtgacaggcttccctgggccccagggccccctgggaaagccaggggctccaggagaacctgggccacaaggccctattggggtaccgggggttcaaggacctcctgggatacccggaattggaaagccaggccaggatgggatcccaggccagccaggatttccaggtggcaaaggggagcaaggactgccagggctaccaggggccccaggccttccagggattgggaaaccaggcttcccaggacccaaaggtgaccggggcatgggaggtgttcctggggctcttggaccaagaggggagaaaggaccaataggttccccaggaatagggggttctccaggagagccaggcctgcctggaatcccaggtcctatgggccctccaggtgctattggttttcctggacccaaaggagaaggtgggattgtagggccacaggggccaccaggtcccaagggtgagccagggcttcaaggcttcccaggaaagccaggtttccttggtgaagtagggcctcctggcatgaggggtttcccaggtcccataggccccaagggggaacatgggcaaaaaggtgtaccaggactccctggtgttccagggcttctcggacctaagggagaaccaggaatcccaggggatcagggtttacagggccccccaggtatcccagggattgggggccctagtggccccattggaccacctgggattccaggccccaaaggggagcctggcctcccagggccccctgggttccctggtatagggaaacccggagtggcaggacttcatggccccccagggaagcctggtgcccttggtcctcaaggccagcctggccttccaggacccccaggccctccaggacctccaggacccccagctgtgatgccccctacaccaccaccccagggagagtatctgccagatatggggctgggaattgatggcgtgaaacccccccatgcctacggggctaagaaaggcaagaatggagggccagcctatgagatgcctgcatttaccgccgagctaaccgcacctttcccaccggtgggggccccagtgaagtttaacaaactgctgtataacggcagacagaactacaacccgcagacaggcatcttcacctgtgaggtccctggtgtctactactttgcataccacgttcactgcaaggggggcaacgtgtgggttgctctattcaagaacaacgagcccgtgatgtacacgtacgacgagtacaaaaagggcttcctggaccaggcatctgggagtgcagtgctgctgctcaggcccggagaccgggtgttcctccagatgccctcagaacaggctgcaggactgtatgccgggcagtatgtccactcctccttttcaggatatttattgtatcccatgtaa

SEQ ID NO: 30 - protein, 744 aa, Homo sapiens
MAVLPGPLQLLGVLLTISLSSIRLIQAGAYYGIKPLPPQIPPQMPPQIPQYQPLGQQVPHMPLAKDGLAMGKEMPHLQYGKEYPHLPQYMKEIQPAPRMGKEAVPKKGKEIPLASLRGEQGPRGEPGPRGPPGPPGLPGHGIPGIKGKPGPQGYPGVGKPGMPGMPGKPGAMGMPGAKGEIGQKGEIGPMGIPGPQGPPGPHGLPGIGKPGGPGLPGQPGPKGDRGPKGLPGPQGLRGPKGDKGFGMPGAPGVKGPPGMHGPPGPVGLPGVGKPGVTGFPGPQGPLGKPGAPGEPGPQGPIGVPGVQGPPGIPGIGKPGQDGIPGQPGFPGGKGEQGLPGLPGAPGLPGIGKPGFPGPKGDRGMGGVPGALGPRGEKGPIGSPGIGGSPGEPGLPGIPGPMGPPGAIGFPGPKGEGGIVGPQGPPGPKGEPGLQGFPGKPGFLGEVGPPGMRGFPGPIGPKGEHGQKGVPGLPGVPGLLGPKGEPGIPGDQGLQGPPGIPGIGGPSGPIGPPGIPGPKGEPGLPGPPGFPGIGKPGVAGLHGPPGKPGALGPQGQPGLPGPPGPPGPPGPPAVMPPTPPPQGEYLPDMGLGIDGVKPPHAYGAKKGKNGGPAYEMPAFTAELTAPFPPVGAPVKFNKLLYNGRQNYNPQTGIFTCEVPGVYYFAYHVHCKGGNVWVALFKNNEPVMYTYDEYKKGFLDQASGSAVLLLRPGDRVFLQMPSEQAAGLYAGQYVHSSFSGYLLYPM

SEQ ID NO: 31 - DNA, 2232 bp, Mus musculus
atggctgtgccaccaggccctctacagctgctgggaatactgttcatcatttccctgaactctgtcagactcattcaggccggtgcctactatggaatcaagcctctgccacctcaaatccctcctcagataccaccacaaattccacagtaccagcccttgggccagcaagtccctcacatgcctttgggcaaagatggcctttccatgggcaaggagatgcctcacatgcagtatggcaaagagtacccatacctcccccaatatatgaaggaaatcccacctgtgccaagaatgggcaaggaagtggtgcccaaaaaaggcaaagaagtaccgttagccagtttgcgcggagaacaaggtccccgtggagaacctggaccaagaggaccacctgggccgccaggtttaccaggtcatggaatgcctggaatcaaaggaaaaccagggccccagggatatccaggaattggaaagcccggtatgcctggaatgcctgggaagccaggagccatgggaatgccaggggcaaaaggcgaaattggacccaaaggggaaatcggacctatgggaatcccagggccacaagggcctccaggacctcatggacttcctggcatcgggaaaccgggtgggccagggttaccagggcaaccaggagcaaaaggtgagagggggcccaaaggaccaccaggacctcccggccttcagggtcccaaaggagagaagggcttcgggatgctgggcttgccaggtctgaagggtcctccaggtatgcatggccctcctggaccagttggactaccgggagtaggaaaaccaggagtgacaggctttcctggaccacagggtcccctggggaagccagggcctccaggggaacctggaccacaaggccttattggtgtgccaggagttcaaggacctcctgggatgcctggagttggaaagccaggtcaggatggatcccgcggccaaccaggatttccgggtggtaaaggggaacaaggattgccagggttgcctggacccccaggcctcccaggggtcggaaagccaggtttcccaggacccaaagggcaccggggtattgggggtgttcctggggttcttgggccgagaggggaaaaaggacccattggtgctcctggaatggggggtccacctggagagccaggcctacccggtatcccgggtcccatgggccctccaggtgctattggtttccctggacccaaaggagaaggtggagttgtaggaccacagggtccaccaggccccaagggagagcctggcctccaaggcttccctgggaagccaggttttcttggtgaagtaggtccccctggcatgaggggtttgcctggtcctataggaccaaagggagaaggtggtcacaaagggttgccagggcttcccggtgttccagggctgcttggacccaaaggagaacctggcatacctggggatcagggtctacagggtcccccagggattccagggatcgtaggacctagtggccctattggaccccctgggattccgggccccaaaggagaaccaggcctcccagggccccctgggttccctggtgtagggaagccaggagtagcaggacttcatgggcccccggggaaacctggtgcccttggtccccaaggccagcctggccttcctggacccccaggtcctccaggacccccaggccctccagctgtgatgcctacaccatcaccccagggagagtatctgccagatatgggactaggaattgatggggtgaaaactccgcatgcctatgcgggcaaaaagggcaaacacggagggccagcctatgagatgcctgcgtttactgccgagctgactgtacctttcccaccggtgggggccccagtgaagtttgacaagctgctctacaacggcagacagaactacaatccgcagacaggcatcttcacctgtgaagtcccgggtgtctactactttgcttatcatgttcactgcaagggaggcaacgtatgggttgctctcttcaagaacaacgagcccatgatgtacacatacgacgagtacaagaagggctttctggaccaagcatctggaagcgcagtactgcttttccttcccggagaccaggtgtttctccaaatcccttcagaacaggctgctggactctatcccgggcaatatgtccactcctccttttcaggatatttattgtatcccatgtaa

SEQ ID NO: 32 - protein, 743 aa, Mus musculus
MAVPPGPLQLLGILFIISLNSVRLIQAGAYYGIKPLPPQIPPQIPPQIPQYQPLGQQVPHMPLGKDGLSMGKEMPHMQYGKEYPYLPQYMKEIPPVPRMGKEVVPKKGKEVPLASLRGEQGPRGEPGPRGPPGPPGLPGHGMPGIKGKPGPQGYPGIGKPGMPGMPGKPGAMGMPGAKGEIGPKGEIGPMGIPGPQGPPGPHGLPGIGKPGGPGLPGQPGAKGERGPKGPPGPPGLQGPKGEKGFGMLGLPGLKGPPGMHGPPGPVGLPGVGKPGVTGFPGPQGPLGKPGPPGEPGPQGLIGVPGVQGPPGMPGVGKPGQDGSRGQPGFPGGKGEQGLPGLPGPPGLPGVGKPGFPGPKGHRGIGGVPGVLGPRGEKGPIGAPGMGGPPGEPGLPGIPGPMGPPGAIGFPGPKGEGGVVGPQGPPGPKGEPGLQGFPGKPGFLGEVGPPGMRGLPGPIGPKGEGGHKGLPGLPGVPGLLGPKGEPGIPGDQGLQGPPGIPGIVGPSGPIGPPGIPGPKGEPGLPGPPGFPGVGKPGVAGLHGPPGKPGALGPQGQPGLPGPPGPPGPPGPPAVMPTPSPQGEYLPDMGLGIDGVKTPHAYAGKKGKHGGPAYEMPAFTAELTVPFPPVGAPVKFDKLLYNGRQNYNPQTGIFTCEVPGVYYFAYHVHCKGGNVWVALFKNNEPMMYTYDEYKKGFLDQASGSAVLLFLPGDQVFLQIPSEQAAGLYPGQYVHSSFSGYLLYPM

SEQ ID NO: 33 - DNA, 618 bp, Homo sapiens
atgttgggagccaagccacactggctaccaggtcccctacacagtcccgggctgcccttggttctggtgcttctggccctgggggccgggtgggcccaggaggggtcagagcccgtcctgctggagggggagtgcctggtggtctgtgagcctggccgagctgctgcaggggggcccgggggagcagccctgggagaggcaccccctgggcgagtggcatttgctgcggtccgaagccaccaccatgagccagcaggggaaaccggcaatggcaccagtggggccatctacttcgaccaggtcctggtgaacgagggcggtggctttgaccgggcctctggctccttcgtagcccctgtccggggtgtctacagcttccggttccatgtggtgaaggtgtacaaccgccaaactgtccaggtgagcctgatgctgaacacgtggcctgtcatctcagcctttgccaatgatcctgacgtgacccgggaggcagccaccagctctgtgctactgcccttggaccctggggaccgagtgtctctgcgcctgcgtcgggggaatctactgggtggttggaaatactcaagtttctctggcttcctcatcttccctctctga

SEQ ID NO: 34 - protein, 205 aa, Homo sapiens
MLGAKPHWLPGPLHSPGLPLVLVLLALGAGWAQEGSEPVLLEGECLVVCEPGRAAAGGPGGAALGEAPPGRVAFAAVRSHHHEPAGETGNGTSGAIYFDQVLVNEGGGFDRASGSFVAPVRGVYSFRFHVVKVYNRQTVQVSLMLNTWPVISAFANDPDVTREAATSSVLLPLDPGDRVSLRLRRGNLLGGWKYSSFSGFLIFPL

SEQ ID NO: 35 - DNA, 639 bp, Mus musculus
acagaagacaggaaaatctgtgagttacagaggcacctagcaaggatggggacggaatggcacaaacccaagctgtccttggccctggtgcttctgaccctggaggctgggtgggctcaagaagggtcagagcctgtcctactggagggggagtgcctggtggtctgtgagccaggccgacctactgcaggaggacctgggggtgcagccctgggagaggcacccccaggacgagtggcgtttgctgcagtccgaagccatcaccatgaaccagcaggagaaaccggcaacggtaccagtggtgccatctacttcgaccaggtactggtcaatgagggcgagggctttgatcggacctcgggctgcttcgtggcccctgtccgtggagtctacagcttccgattccacgtggtcaaggtgtacaaccgccaaactgtccaggtgagtctgatgctgaacacatggccggtcatctcagcctttgcaaatgaccctgatgtgacccgagaggcagccacaagctctgtgctgcttcccctggacccgggggaccgtgtgtccctgcgcctgcgtcgggggaatcttctgggtggctggaagtattcgagcttctcaggcttcctcatcttcccactctga

SEQ ID NO: 36 - protein, 213 aa, Mus musculus
MTEDRKICELQRHLARMGTEWHKPKLSLALVLLTLEAGWAQEGSEPVLLEGECLVVCEPGRPTAGGPGGAALGEAPPGRVAFAAVRSHHHEPAGETGNGTSGAIYFDQVLVNEGEGFDRTSGCFVAPVRGVYSFRFHVVKVYNRQTVQVSLMLNTWPVISAFANDPDVTREAATSSVLLPLDPGDRVSLRLRRGNLLGGWKYSSFSGFLIFPL

(19) United States
(12) Patent Application Publication
(10) Pub. No.: US 2003/0134328 A1
(43) Pub. Date: Jul. 17, 2003

(54) Mammalian genes; related reagents

(76) Inventors: Beth E. Basham, San Jose, CA (US); Ian Forsythe, Edmonton (CA); Daniel M. Gorman, Newark, CA (US); Jeanine D. Mattson, San Francisco, CA (US); Mehrdad Moshrefi, Mountain View, CA (US); Christi Parham, San Francisco, CA (US)

Correspondence Address: DNAX RESEARCH, INC., LEGAL DEPARTMENT, 901 CALIFORNIA AVENUE, PALO ALTO, CA, 94304, US

(21) Appl. No.: 10/236,055

(22) Filed: Sep. 4, 2002

Related U.S. Application Data
(60) Provisional application No. 60/317,988, filed on Sep. 6, 2001.

(51) Int. Cl. G01N 33/53; C07H 21/04; A61K 39/395; C12P 21/02; C12N 5/06; C07K 14/47; C07K 16/44

(52) U.S. Cl. 435/7.1; 435/69.1; 435/320.1; 435/325; 530/350; 530/388.15; 424/130.1; 536/23.5

(57) ABSTRACT

Purified nucleic acids encoding C1QSF3 from a mammal, purified C1QSF3 proteins, specific antibodies thereto, and related reagents are provided. Methods of using these molecules and reagents are also provided.

This application claims benefit of U.S. Provisional Patent Application No. 60/317,988, filed Sep. 6, 2001.

### FIELD OF THE INVENTION

The present invention pertains to compositions related to proteins which function in controlling activation and expansion of mammalian cells, e.g., cells of a mammalian immune system. In particular, it provides purified genes, proteins, antibodies, and related reagents useful, e.g., to regulate activation, development, differentiation, and function of various cell types, including hematopoietic cells.

### BACKGROUND OF THE INVENTION

Proteins of the Tumor Necrosis Factor (TNF) and the complement C1q families share structural and functional similarities. The TNF and C1q families share certain conserved amino acid residues, a common folding topology, i.e., globular C1q (gC1q) domain, and similar gene structures (Kishore and Reid (1999) Immunopharmacol. 42:15-21). Both groups of proteins appear to play major roles in immunity as well as in energy homeostasis.

Typical TNF family molecules involved in immunity include CD40L, TNFα, and FAS ligand (see, e.g., Tracey and Cerami (1994) Annu. Rev. Med. 45:491-503). CD40L knockout mice are defective in antibody class switching and lack all immunoglobulins except IgM in the T-dependent humoral response (see, e.g., Renshaw, et al. (1994) J. Exp. Med. 180:1889-1900). Similarly, C1q knockout mice also exhibit defects in class switching (see, e.g., Shapiro and Scherer (1998) Curr. Biol. 8:335-338). This suggests that these structurally related molecules may play similar roles.

Like many complement proteins, TNFα is produced in response to infection and effects multiple responses, including inflammation, cell proliferation, and cell death acting via TNF receptors (see, e.g., Tracey and Cerami, supra). TNFα has also been shown to regulate the expression levels of some downstream components of the complement system (see, e.g., Kulics, et al. (1994) Immunology 82:509-515; and Kawakami, et al. (1997) Cancer Letts. 116:21-26). TNF also plays a role in energy homeostatis, where it is implicated in cachexia, obesity, and insulin resistance (see, e.g., Hotamislgil and Spiegelman (1994) Diabetes 1271-1278; and Teoman, et al. (1997) 389:610-614). It also is a major secretory product of adipocytes (see, e.g., Hotamislgil and Speigelman, supra.).

Similar activities have been observed for C1q family proteins. For example, ACRP30 is made exclusively in adipocytes and its expression is dysregulated in various forms of obesity. ACRP30 secretion is acutely stimulated by insulin and repressed by chronically elevated levels of insulin (see, e.g., Shapiro and Scherer, supra). Another C1q-like molecule, the Hib27 molecule from Siberian chipmunks, also seems to be involved in energy homeostasis, as its expression is specifically extinguished during hibernation (see, e.g., Takamatsu, et al. (1993) Mol. Cell. Biol. 13:1516-1521). The functional connection between adipose tissue and immunity has been noted before: adipocytes secrete high levels of complement factors C3 and B, and provide the unique site of synthesis for complement factor D (see, e.g., Flier, et al. (1987) Science 237:405-408).

C1q is the first subcomponent of the C1 complex of the classical pathway of complement activation. C1q plays a key role in the recognition of immune complexes. The molecule consists of several structural chains and domains, including a C-terminal globular region (gC1q domain) of approximately 135 residues (see, e.g., Sellar et al. (1991) Biochem. J. 274:481-490). Several molecules that contain the gC1q domain have been identified and placed in a new category of structurally similar molecules known as the C1q/TNF superfamily. Included in this group of molecules are precerebellin, multimerin, EMILIN, C1qA, C1qB, C1qC, Acrp-30, HP, Type X collagen, Type VIII collagen, and saccular collagen.

Recently, it has been observed that C1q containing molecules are seen in Lewy bodies, oligodendroglia, and the substantia nigra in cells from Parkinson's disease brain tissue. Also present in these tissues are increased levels of TNFCC and other cytokines (see, e.g., McGeer, et al. (2001) BC Med. J. 43:138-141). It is known that in patients suffering from Parkinson's disease, there is an increase in microglial cells, which are the main immune defense in the brain. C1q containing proteins can activate these cells (see, e.g., McGeer, et al. supra.) Further studies have revealed that TNF and C1Q containing molecules are involved in the disease progression of a number of inflammatory diseases, e.g., inflammatory bowel disease, Crohn's disease, skin inflammation, psoriasis, and Hashimoto's thyroiditis (See, e.g., Federici, et al. (2002) J. Immunol. 169:434-442; Suzuki, et al. (2001) J. Exp. Med. 193:471-482; Aust, et al. (1996) Clin. Exp. Immunol. 105:148-154).

A number of pathways that regulate inflammation or energy metabolism have been identified, where these pathways involve proteins of the TNF/C1q superfamily. However, the molecules and interactions responsible for inflammation and energy metabolism are only partially understood. The present invention fulfills this need by identifying a new member of the TNF/C1q superfamily.

### BRIEF DESCRIPTION OF THE DRAWING

FIGS. 1A-D shows an alignment of TNF/C1Q superfamily members and the molecules of the present invention. Boxes indicate β-strands, and shaded areas are structural domains as predicted by XTAL.

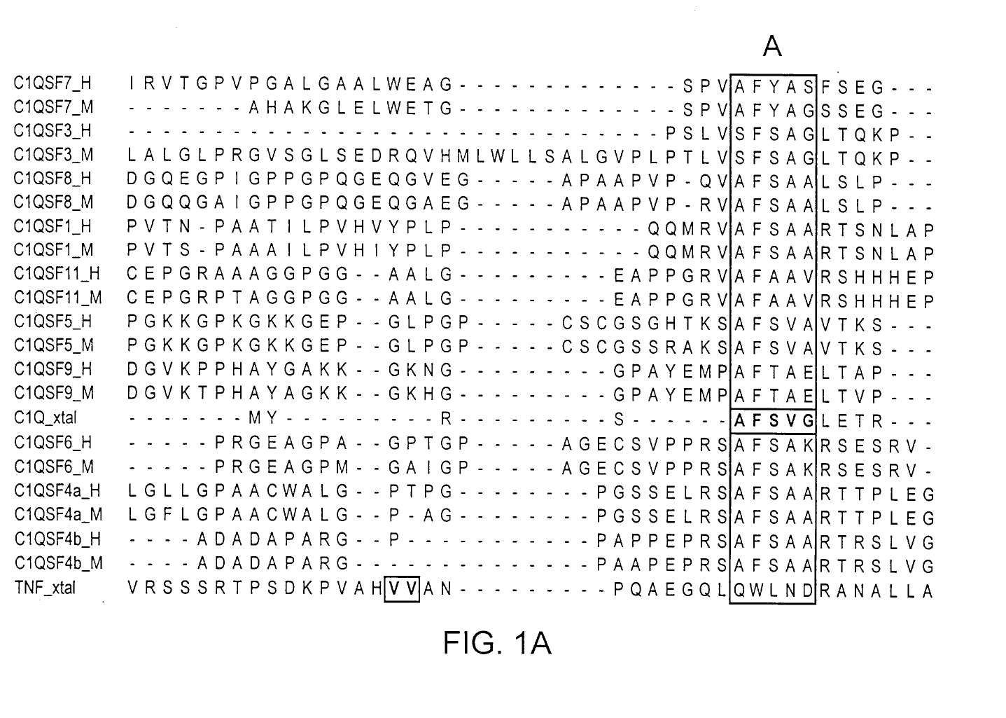

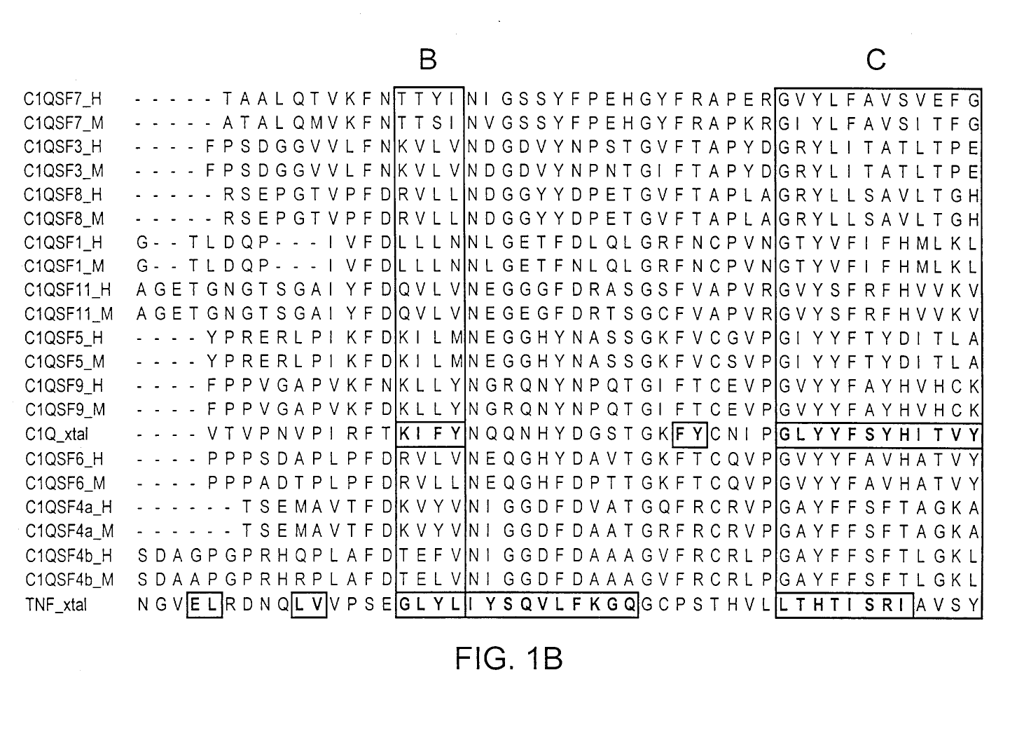

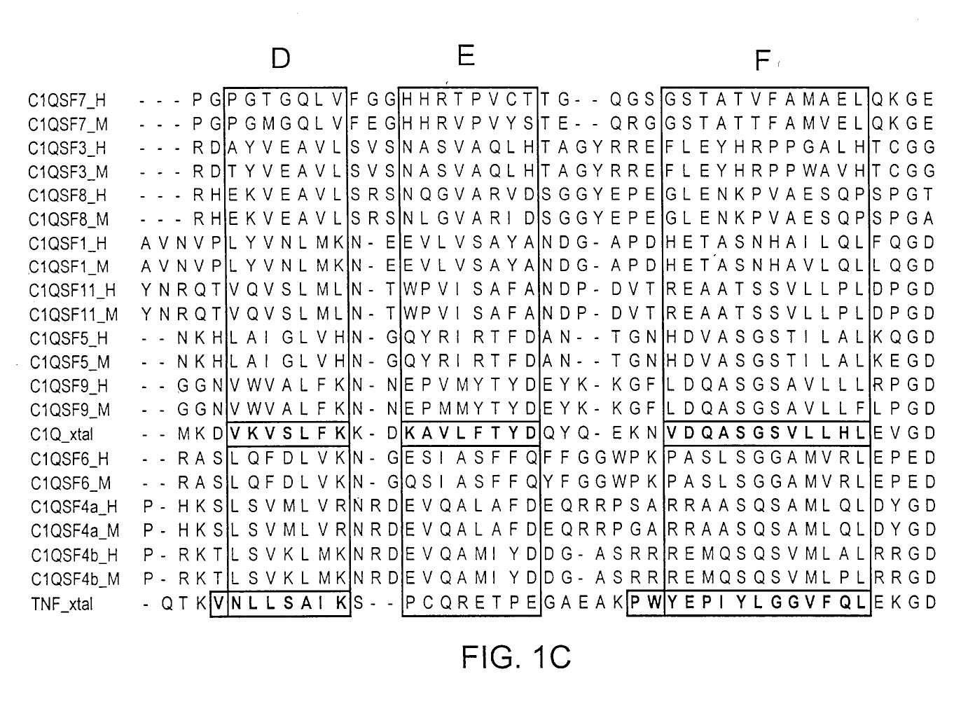

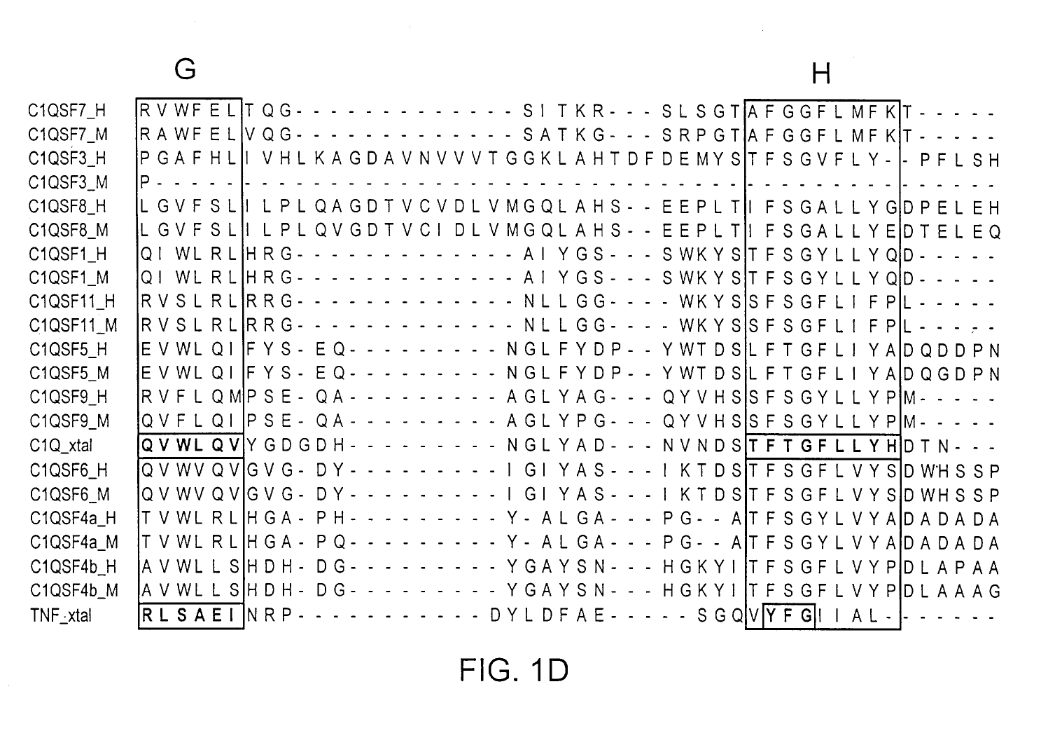

### SUMMARY OF THE INVENTION

The present invention is based, in part, upon the discovery of a molecule that has structural similarity to TNF/C1q superfamily members. In particular, it provides polypeptides, polynucleotides, binding compositions, methods of production, methods of use, and kits.

The present invention provides an isolated nucleic acid encoding a polypeptide comprising SEQ ID NO:10 or 12, or an antigenic fragment thereof. Also provided is an isolated nucleic acid which hybridizes to a nucleic acid encoding a polypeptide comprising SEQ ID NO:10 or 12, or an antigenic fragment thereof, under stringent conditions. The invention encompasses an isolated nucleic acid comprising SEQ ID NO:9 or 11. Additionally provided is an expression or replicating vector comprising a nucleic acid encoding a polypeptide comprising SEQ ID NO:10 or 12, or an antigenic fragment thereof. The invention also encompasses a host cell comprising an expression or replicating vector comprising a nucleic acid encoding a polypeptide comprising SEQ ID NO:10 or 12, or an antigenic fragment thereof.

The invention provides an isolated polypeptide comprising SEQ ID NO:10 or 12, or an antigenic fragment thereof. The invention also provides an isolated polypeptide comprising SEQ ID NO:10 or 12, or an antigenic fragment thereof, further comprising a fusion polypeptide or peptide.

The invention provides a binding composition which specifically binds to a polypeptide comprising SEQ ID NO:10 or 12, or an antigenic fragment thereof. The invention also encompasses a binding composition which specifically binds to an isolated polypeptide comprising SEQ ID NO:10 or 12, or an antigenic fragment thereof, where the binding composition may comprise an antigen binding site of an antibody, or where the antigen binding site may be a polyclonal antibody, monoclonal antibody, humanized antibody, Fab fragment, F(ab′)₂ fragment, or Fv fragment, or where the antigen binding site is detectably labeled.

Also encompasses is a binding composition which specifically binds to a polypeptide comprising SEQ ID NO:10 or 12, or an antigenic fragment thereof, in conjunction with an acceptable carrier. Also provided is a kit comprising a substantially pure polypeptide comprising SEQ ID NO:10 or 12, or an antigenic fragment thereof, a binding composition which specifically binds the polypeptide; or a nucleic acid encoding the polypeptide.

Additionally, the invention provides a method of producing a polypeptide comprising SEQ ID NO:10 or 12, or an antigenic fragment thereof, comprising culturing a host cell comprising an expression or replicating vector comprising a nucleic acid encoding a polypeptide comprising SEQ ID NO:10 or 12 under conditions suitable for expression of the polypeptide, and isolating or purifying the polypeptide. Also provided is a method of modulating the activity of a cell comprising contacting the cell with a binding composition which specifically binds to the polypeptide comprising SEQ ID NO:10 or 12, or an antigenic fragment thereof.

The invention encompasses a method of treating a subject suffering from an inflammatory condition comprising administering an effective amount of an agonist or antagonist of a polypeptide comprising SEQ ID NO:10 or 12, or an antigenic fragment thereof. Also encompassed is a method of treating a subject suffering from an inflammatory condition comprising administering an effective amount of an agonist or antagonist of a polypeptide comprising SEQ ID NO:10 or 12, or an antigenic fragment thereof, where the agonist or antagonist is a binding composition which specifically binds to a polypeptide comprising SEQ ID NO:10 or 12, or an antigenic fragment thereof.

### DETAILED DESCRIPTION

As used herein, including the appended claims, the singular forms of words such as “a,” “an,” and “the,” include their corresponding plural references unless the context clearly dictates otherwise.

All references cited herein are incorporated herein by reference to the same extent as if each individual publication or patent application was specifically and individually indicated to be incorporated by reference.

I. Definitions.

“Activity” of a molecule may describe or refer to binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, including selenomethionine, as well as those amino acids that are modified after incorporation into a polypeptide, e.g., hydroxyproline, γ-carboxyglutamate, O-phosphoserine, and cystine. Amino acid analogs refers to compounds that have the same basic chemical structure as a naturally occurring amino acid, i.e., an alpha-carbon that is bound to a hydrogen, a carboxyl group, an amino group, and an R group, e.g., homoserine, norleucine, methionine sulfoxide, methionine methyl sulfonium. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by their one-letter symbols.

“Antibody” refers to a polypeptide comprising a framework region from an immunoglobulin gene or fragments thereof that specifically binds and recognizes an antigen. The immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region.

“Fv” fragment comprises a dimer of one heavy chain and one light chain variable domain in tight association with each other. A single variable domain (or half of an Fv comprising only three CDRs specific for an antigen) has the ability to recognize and bind antigen, although at a lower affinity than the entire binding site (Paul (ed) (1993) Fundamental Immunology, Third Ed., Raven Press, New York).

The term “monoclonal antibody” (mAb) refers to an antibody obtained from a population of substantially homogeneous antibodies, i.e., the individual antibody polypeptides comprising the population are identical except for possible naturally occurring mutations in the polypeptide chain that may be present in minor amounts. The term “monoclonal antibody” does not suggest any characteristic of the oligosaccharide component, or that there is homogeneity or heterogeneity with regard to oligosaccharide component. Monoclonal antibodies are highly specific, being directed against a single antigenic site or epitope. In contrast to conventional (polyclonal) antibody preparations which typically include different antibodies directed against different epitopes, each mAb is directed against a single determinant on the antigen. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. “Monoclonal antibodies” also include clones of antigen-recognition and binding-site containing antibody fragments, such as those derived from phage antibody libraries.

“Diabodies” refers to a fragment comprising a heavy chain variable domain (V_{H}) connected to a light chain variable domain (V_{L}) (Hollinger, et al. (1993) Proc. Natl. Acad. Sci. USA 90:6444-6448).

“Binding composition” refers to a molecule, small molecule, macromolecule, antibody, or a fragment or analog, thereof, which is capable of binding to a target. “Binding composition” also may refer to a complex of molecules, e.g., a non-covalent complex, to an ionized molecule, and to a covalently or non-covalently modified molecule, e.g., modified by phosphorylation, acylation, cross-linking, or cyclization, which is capable of binding to a target. “Binding composition” may also refer to a molecule capable of binding to a target in combination with a stabilizer, excipient, salt, buffer, solvent, or additive. “Binding” may be defined as an association of the binding composition with a target where the normal Brownian motion of the binding composition is prevented or impaired, in cases where the binding composition can be dissolved or suspended in solution.

“Cell line” refers to a population of cells capable of continuous or prolonged growth and division in vitro. Often, cell lines are clonal populations derived from a single progenitor cell. Spontaneous or induced changes can occur in the genome or can occur during storage or transfer of one or more cells present in the population of cells. Therefore, cells derived from the cell line referred to may not be precisely identical to the ancestral cells or cultures, and the cell line referred to includes such variants. The term “cell line” also includes immortalized cells (U.S. Pat. No. 6,090,611 issued to Covacci, et al.).

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical nucleic acid sequences. Because of the degeneracy of the genetic code, a large number of functionally identical nucleic acids encode any given protein.

As to amino acid sequences, one of skill will recognize that individual substitutions, deletions or additions to a nucleic acid, peptide, polypeptide, or protein sequence which alters, adds or deletes a conserved amino acid or a small percentage of amino acids in the encoded sequence is a “conservatively modified variant.” Conservative substitution tables providing functionally similar amino acids are well known in the art. An example of a conservative substitution is the exchange of an amino acid in one of the following groups for another amino acid of the same group (U.S. Pat. No. 5,767,063 issued to Lee, et al.; Kyte and Doolittle (1982) J. Mol. Biol. 157:105-132):

(1) Hydrophobic: Norleucine, Ile, Val, Leu, Phe, Cys, or Met;

(2) Neutral hydrophilic: Cys, Ser, Thr;

(3) Acidic: Asp, Glu;

(4) Basic: Asn, Gln, His, Lys, Arg;

(5) Residues that influence chain orientation: Gly, Pro;

(6) Aromatic: Trp, Tyr, Phe;

(7) Small amino acids: Gly, Ala, Ser.

“Exogenous” refers to substances that are produced outside an organism or cell, depending on the context. “Endogenous” refers to substances that are produced within a cell or organism, depending on the context.

An “expression vector” is a nucleic acid construct, generated recombinantly or synthetically, with a series of specified nucleic acid elements that permit transcription of a particular nucleic acid. Typically, the expression vector includes a nucleic acid to be transcribed operably linked to a promoter.

“Fusion protein or polypeptide” refers to a polypeptide chain synthesized from a nucleic acid, where the nucleic acid comprises an open reading frame encoding two or more polypeptide or peptide sequences, where the two or more nucleic acid sequences generally do not occur together to encode a single open reading frame. One or all of the nucleic acids encoding the fusion protein may be of synthetic origin. The fusion protein may be synthesized by recombinant or synthetic means, or it may occur naturally.

“Gene expression” refers to transcription or translation, depending on the context. In transcription, mRNA is expressed from a gene. In translation, a polypeptide is expressed from mRNA.

An “immunoassay” is an assay that uses an antibody or antibody fragment to specifically bind an antigen. The immunoassay is characterized by the use of specific binding properties of a particular antibody to isolate, target, or quantify the antigen.

“Inhibitors” or “antagonists” and “activators” or “agonists” refer to inhibitory or activating molecules, respectively, identified using in vitro or in vivo assays, e.g., for the activation of a ligand, a receptor, a cofactor, or a gene. A “modulator” of gene activation or of protein activity is a molecule that is an inhibitor or an activator of a given gene or protein. The modulator may act alone, or it may use or require a cofactor, e.g., a protein, metal ion, or small molecule. Inhibitors are compounds that decrease, block, prevent, delay activation, inactivate, desensitize, or down regulate a gene or protein. Activators are compounds that increase, activate, facilitate, enhance activation, sensitize, or up regulate a gene or protein. To examine the extent of inhibition, samples or assays comprising a given gene or protein are treated with a potential activator or inhibitor and are compared to control samples without the inhibitor. Control samples (untreated with inhibitors) are assigned a relative activity value of 100%. Inhibition is achieved when the activity value relative to the control is about 90%, generally at least 85%, more generally at least 80%, preferably 75%, more preferably 70%, often 65%, more often 60%, typically 55%, more typically 50%, ordinarily 45%, ordinarily at least 40%, usually 35%, most usually 30%, most usually at least 25%, and most preferably less than 25%. Activation is achieved when the activity value relative to the control is about 110%, generally 120%, more generally 140%, more generally at least 160%, preferably 180%, more preferably 2-fold, often 2.5-fold, more often 5-fold, typically 10-fold, more typically 20-fold, usually 40-fold, and most usually over 40-fold higher. An “agonist” is a compound that interacts with a target or that can cause an increase in the activation of the target. An “antagonist” is a compound that opposes the actions of an agonist. An agonist prevents, inhibits, or neutralizes the activity of an agonist.

A composition that is “labeled” is detectable, either directly or indirectly, by spectroscopic, photochemical, biochemical, immunochemical, isotopic, or chemical means. For example, useful labels include ³²P, ³³P, ³⁵S, ¹⁴C, ³H, 1251, stable isotopes, fluorescent dyes, fluorettes (Rozinov and Nolan (1998) Chem. Biol. 5:713-728), electron-dense reagents, enzymes and/or substrates, e.g., as used in enzyme-linked immunoassays as with those using alkaline phosphatase or horse radish peroxidase. The label or detectable moiety is typically bound, either covalently, through a linker or chemical bound, or through ionic, van der Waals or hydrogen bonds to the molecule to be detected. “Radiolabeled” refers to a compound to which a radioisotope has been attached through covalent or non-covalent means. A “fluorophore” is a compound or moiety that absorbs light energy of one wavelength and emits light energy of a second, longer wavelength.

“Ligand” refers to an entity that specifically binds to a polypeptide or a complex of more than one polypeptide. A “ligand binding domain” is a region of a polypeptide that is able to bind to the entity. A ligand may be a soluble protein, a membrane-associated protein, or an integral membrane-bound protein. Where a ligand binds to a receptor, the question of which molecule is the ligand and which molecule is the receptor may be determined on a case-by-case basis. Generally, where the binding event results in cell signaling, a molecule that is constitutively bound to the cell that responds to the signal may be considered to be part of the receptor, and not part of the ligand. A freely diffusable and water-soluble entity that is involved in ligand/receptor interactions is usually a ligand, not a receptor. Ligands may also comprise oligosaccharides, lipids, and the like.

“Nucleic acid” refers to deoxyribonucleotides or ribonucleotides and polymers thereof, including single stranded and double stranded forms. The term encompasses nucleic acids containing nucleotide analogs or modified backbone residues or linkages. Examples of such analogs, e.g., phosphorothioates, phosphoramidates, and peptide-nucleic acids (PNAs).

A particular nucleic acid sequence also implicitly encompasses degenerate codon substitutions, i.e., different codons that code for the same amino acid, nucleotide base substitutions that code for conservative amino acid changes, and complementary sequences. The term nucleic acid may be used to refer, e.g., to a gene, cDNA, mRNA, oligonucleotide, and polynucleotide. A particular nucleic acid sequence also implicitly encompasses allelic variant, splice variants, and genetic mutations.

Substantial homology in the nucleic acid sequence comparison context means either that the segments, or their complementary strands, when compared, are identical when optimally aligned, with appropriate nucleotide insertions or deletions, in at least about 50% of the nucleotides, generally at least about 58%, ordinarily at least about 65%, often at least about 71%, typically at least about 77%, usually at least about 85%, preferably at least about 95 to 98% or more, and in particular embodiments, as high as about 99% or more of the nucleotides. Typically, selective hybridization will occur when there is at least about 55% homology over a stretch of at least about 30 nucleotides, preferably at least about 75% over a stretch of about 25 nucleotides, and most preferably at least about 90% over about 20 nucleotides. See, Kanehisa (1984) Nuc. Acids Res. 12:203-213. The length of homology comparison, as described, may be over longer stretches, and in certain embodiments will be over a stretch of at least about 17 nucleotides, usually at least about 28 nucleotides, typically at least about 40 nucleotides, and preferably at least about 75 to 100 or more nucleotides.

“Peptide” refers to a short sequence of amino acids, where the amino acids are connected to each other by peptide bonds. A peptide may occur free or bound to another moiety, such as a macromolecule or a polypeptide. Where a peptide is incorporated into a polypeptide chain, the term “peptide” may still be used to refer specifically to the short sequence of amino acids. A “peptide” may be connected to another moiety by way of a peptide bond, or by way of another type of linkage. A peptide is at least two amino acids in length. A peptide is usually less than about 25 amino acids in length, where the maximal length is a function of custom or context. The terms “peptide” and “oligopeptide” may be used interchangeably.

The term “protein” generally refers to the sequence of amino acids comprising a polypeptide chain. Protein may also refer to a three dimensional structure of the polypeptide. “Denatured protein” refers to a partially or totally denatured polypeptide, having some residual three dimensional structure or, alternatively, an essentially random three dimensional structure. The invention also encompasses polypeptide variants, e.g., involving glycosylation, phosphorylation, sulfation, disulfide bond formation, deamidation, isomerization, cleavage points in signal or leader sequence processing, covalent and non-covalently bound cofactors, and the like. The formation of disulfide linked proteins and variations thereof is described, e.g., see Woycechowsky and Raines (2000) Curr. Opin. Chem. Biol. 4:533-539; Creighton, et al. (1995) Trends Biotechnol. 13:18-23.

By “purified” and “isolated” is meant, when referring to a polypeptide, that the polypeptide is present in the substantial absence of the other biological macromolecules. The term “purified” as used herein means typically about 70%, more typically 75%, at least 80%, ordinarily 85%, more ordinarily 90%, preferably 95%, more preferably 98% by weight, or greater, of biological macromolecules present. The weights of water, buffers, salts, detergents, reductants, protease inhibitors, stabilizers, excipients, and other small molecules, especially those having a molecular weight of less than 1000, are generally not used in the determination of polypeptide purity (U.S. Pat. No. 6,090,611). Purity and homogeneity are typically determined using methods well known in the art (Scopes (1994) Protein Purification: Principles and Practice, Springer-Verlag, NY, N.Y.; Cunico, Gooding, and Wehr (1998) Basic HPLC and CE of Biomolecules, Bay Biological Laboratory, Inc. Hercules, Calif.).

“Recombinant” when used with reference, e.g., to a nucleic acid, cell, virus, plasmid, vector, or the like, indicates that these have been modified by the introduction of an exogenous, non-native nucleic acid or the alteration of a native nucleic acid, or have been derived from a recombinant nucleic acid, cell, virus, plasmid, or vector. Recombinant protein refers to a protein derived from a recombinant nucleic acid, virus, plasmid, vector, or the like.

“Soluble receptor” refers to receptors that are water-soluble and occur, e.g., in extracellular fluids, intracellular fluids, or weakly associated with a membrane. Soluble receptor also refers to receptors that are released from tight association with a membrane, e.g., by limited cleavage. Soluble receptor further refers to receptors that are engineered to be water soluble. See. e.g., Monahan, et al. (1997) J. Immunol. 159:4024-4034; Moreland, et al. (1997) New Engl. J. Med. 337:141-147; Borish, et al. (1999) Am. J. Respir. Crit. Care Med. 160:1816-1823; Uchibayashi, et al. (1989) J. Immunol. 142:3901-3908. The invention contemplates use of a soluble receptor to C1QSF3 for use in modulating the activity of C1QSF3, e.g., in the treatment of inflammation.

The phrase “specifically” or “selectively” binds, when referring to a ligand/receptor, antibody/antigen, or other binding pair, refers to a binding reaction which is determinative of the presence of the protein in a heterogeneous population of proteins and other biologics. Thus, under designated conditions, a specified ligand binds to a particular receptor and does not bind in a significant amount to other proteins present in the sample. The contemplated antibody of the invention binds to its antigen, or a variant or mutein thereof, with an affinity that is about ten times greater, more preferably 20-times greater, and still more preferably 100-times greater than the affinity with any other antibody. In a preferred embodiment the antibody will have an affinity which is greater than about 10⁹ liters/mol, as determined, for example, by Scatchard analysis (Munsen, et al. (1980) Analyt. Biochem. 107:220-239).

“Stringent conditions” are those that (1) employ low ionic strength and high temperature for washing, for example, 0.015 M sodium chloride/0.0015 M sodium citrate/0.1% sodium dodecyl sulfate at 50° C.; (2) employ during hybridization a denaturing agent, such as formamide, for example, 50% (vol/vol) formamide with 0.1% bovine serum albumin/0.1% Ficoll® (Sigma, St. Louis, Mo.)/0.1% polyvinylpyrrolidone/50 mM sodium phosphate buffer at pH 6.5 with 750 mM sodium chloride, 75 mM sodium citrate at 42° C.; (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 ng/ml), 0.1% SDS, and 10% dextran sulfate at 42° C., with washes at 42° C. in 0.2× SSC and 0.1% SDS; or (4) employ a buffer of 10% dextran sulfate, 2× SSC (sodium chloride/sodium citrate), and 50% formamide at 55° C., followed by a high-stringency wash consisting of 0.1×SSC containing EDTA at 55° C. (U.S. Pat. No. 6,387,657 issued to Botstein, et al.). “Moderately stringent conditions” are described in Sambrook et al., Molecular Cloning: A Laboratory Manual (New York: Cold Spring Harbor Laboratory Press, 1989), and include the use of a washing solution and hybridization conditions (e.g., temperature, ionic strength, and percent SDS) less stringent than described above.

“Treatment” refers to both therapeutic treatment and prophylactic or preventative measures.

II. General.

The present invention provides amino acid sequences and DNA sequences encoding mammalian C1QSF3, e.g., human C1QSF3 and murine C1QSF3. Human and murine C1QSF3 polynucleotide and polypeptide sequences are represented by SEQ ID NOs:9 and 10 (human) and SEQ ID NOs:11 and 12 (murine). Regions of homology between C1QSF3 and other members of the C1QSF family are shown (Table 1). Table 1 summarizes approximate residue boundaries of the structural motifs or characteristics of the C1QSF family of polypeptides.

TABLE 1

| | SEQ ID | Leader | C1Q | TNF | C1Q/TNF | Collagen |
|---|---|---|---|---|---|---|
| C1QSF | NO: | sequence | Domain | Domain | Domain | Domains |
| Human C1QSF1 | 2 | | 284-420 | | | |
| Murine C1QSF1 | 4 | | 273-409 | | | |
| Human C1QSF 2/5 | 6 | 1 to 16 | 188-323 | | | 83-187 |
| Murine C1QSF 2/5 | 8 | 1 to 24 | 152-286 | | | 40-151 |
| Human C1QSF3 | 10 | 1 to 20 | 908-952 | | 1011-1047 | |
| Murine C1QSF3 | 12 | 1 to 18 | 50-95 | | | |
| Human C1QSF4 | 14 | 1 to 16 | 23-311 | | | |
| Murine C1QSF4 | 16 | 1 to 16 | 22-309 | | | |
| Human C1QSF6 | 18 | 1 to 15 | 97-234 | | | 36-95 |
| Murine C1QSF6 | 20 | 1 to 15 | 97-234 | | | 36-95 |
| Human C1QSF7 | 22 | 1 to 22 | 827-873 | 911-915 | 916-946 | |
| Murine C1QSF7 | 24 | 1 to 22 | 623-657 | | | |
| Human C1QSF8 | 26 | | 768-819 | | 878-917 | 713-767 |
| Murine C1QSF8 | 28 | | 652-703 | | 758-796 | 602-651 |
| Human C1QSF9 | 30 | 1 to 27 | 609-744 | | | 147-205; |
| | | | | | | 208-266; |
| | | | | | | 275-571. |
| Murine C1QSF9 | 32 | 1 to 27 | 608-743 | | | 118-175; |
| | | | | | | 185-243; |
| | | | | | | 255-313; |
| | | | | | | 316-374; |
| | | | | | | 381-440; |
| | | | | | | 444-503; |
| | | | | | | 513-571. |
| Human C1QSF11 | 34 | 1 to 32 | 71-205 | | | |
| Murine C1QSF11 | 36 | 1 to 40 | 79-213 | | | |

Members of the C1q/TNF family appear to function, e.g., in collagen physiology, mammalian hibernation, adipose tissue physiology, neurogenesis, synapse formation, platelet physiology, skeletal physiology and growth plate formation, hormone-transport and binding, energy homeostasis and insulin response, elastin deposition, inflammation, adaptive immunity, and apoptosis. See, e.g., Kishore and Reid (2000) Immunopharmacology 49:159-170 and Doliana, et al. (2001) J. Biol. Chem. 276:12003-12011.

III. Analogs of C1QSF3 and Binding Compositions Thereto.

This invention also encompasses proteins or peptides having substantial amino acid sequence identity with the amino acid sequence of the C1QSF3, antigenic fragments thereof, and binding composition thereto, including polymorphic variants, allelic variants, and variants due to mutations and alternative splicing. The invention also contemplates C1QSF3 species that are modifed by recombinant or chemical techniques.

Mutagenesis can be conducted by making amino acid insertions or deletions. Substitutions, deletions, insertions, or any combinations may be generated to arrive at a final construct. Insertions include amino- or carboxy-terminal fusions. Random mutagenesis can be conducted at a target codon and the expressed mutants can then be screened for the desired activity. Methods for making substitution mutations at predetermined sites in DNA having a known sequence are well known in the art, e.g., by M13 primer mutagenesis or polymerase chain reaction (PCR) techniques.

Covalent derivatives can be prepared by linkage of functionalities to groups which are found in C1QSF amino acid side chains or at the N- or C-termini, e.g., by standard means. See, e.g., Lundblad and Noyes (1988) Chemical Reagents for Protein Modification, vols. 1-2, CRC Press, Inc., Boca Raton, Fla.; Hugli (ed.) (1989) Techniques in Protein Chemistry, Academic Press, San Diego, Calif.; and Wong (1991) Chemistry of Protein Conjugation and Cross Linking, CRC Press, Boca Raton, Fla.

Fusion polypeptides comprising C1QSF3, or a fragment thereof, as well as the nucleic acids encoding them, can be made by a number of methods (Sambrook, et al. (1989) Molecular Cloning: A Laboratory Manual (2d ed.), vols. 1-3, Cold Spring Harbor Laboratory; and Ausubel, et al. (eds.) (1993) Current Protocols in Molecular Biology, Greene and Wiley, NY). The contemplated fusion proteins include those containing an affinity tag, a reporter polypeptide or enzyme, a receptor-binding segment, a linker, an immune domain, or a cytokine or cytokine receptor. The contemplated embodiments include, e.g., luciferase, bacterial β-galactosidase, trpE, Protein A, β-lactamase, alpha amylase, alcohol dehydrogenase, yeast alpha mating factor, a FLAG sequence, and a His6 sequence. See, e.g., Godowski, et al. (1988) Science 241:812-816; Rais-Beghdadi, et al. (1998) Appl. Biochem. Biotechnol. 74:95-103; Dull, et al., U.S. Pat. No. 4,859,609). “Linkers” are generally short, self-complementary oligomers which connect longer nucleic acid sequences. Linkers may or may not maintain the open reading frame between the connected nucleic acid sequences, may or may not contain a restriction site, and may be blunt-ended or contain overhanging bases, depending on the use and context. See, e.g., Catalogue (2002) New England Biolabs, Inc., Beverly, Mass., pp. 142-143.

The invention contemplates C1QSF3 polypeptides modified by changes in oligosaccharide identity or content, and location of oligosaccharides on the polypeptide. See, e.g., Elbein (1987) Ann. Rev. Biochem. 56:497-534; Summers (1988) Bio/Technology 6:47:55; and Kaufman (1990) Meth. Enzymol. 185:487/511. Also embraced are versions of the peptides and polypeptides with the same primary amino acid sequence which have other minor modifications, including phosphorylated amino acid residues, e.g., phosphotyrosine, phosphoserine, or phosphothreonine. The invention also contemplates modification by a moiety comprising a lipid, e.g., phosphatidyl inositol (Low (1989) Biochim. Biophys. Acta 988:427-454; Tse, et al. (1985) Science 230:1003-1008; and Brunner, et al. (1991) J. Cell Biol. 114:1275-1283).

The invention further contemplates immobilization, e.g., to a bead, magnetic bead, slide, microarray, fabric, or device such as a lab-on-a-chip. The invention contemplates immobilized nucleic acids, polypeptides, peptides, antibodies and antibody fragments, as well as other reagents.

IV. Screening for C1QSF3 expression and for therapeutic agents.

Cells or animals may be screened for expression of a C1QSF3 gene. Levels of mRNA may be measured by techniques using hybridization, such as Northern blotting or the molecular beacon technique (Liu, et al. (2002) Analyt. Biochem. 300:40-45), or techniques that combine reverse transcription and the polymerase chain reaction (RT-PCR). See, e.g., Huang, et al. (2000) Cancer Res. 60:6868-6874). PCR product can be measured by incorporated radiolabel, or by electrophoresis followed by staining with a dye, such as ethidium bromide. Alternatively, PCR product can be measured during each cycle of the PCR reaction, e.g., by means of TaqMan® (PE Applied Biosystems, Foster City, Calif.) probes or by SYBR Green I® (Molecular Probes, Eugene, Oreg.) (Wittwer, et al. (1997) Biotechniques 22:130-138; Schmittgen, et al. (2000) Analyt. Biochem. 285:194-204). The TaqMan® technique, and similar techniques, rely on nuclease digestion of a probe, where digestion releases a fluorescing dye, where release results in an increase in fluorescence (Heid, et al. (1996) Genome Res. 6:989-994).

Microarrays of nucleic acids may be used for screening (Ausubel, et al. (2001) Curr. Protocols Mol. Biol., Vol. 4, John Wiley and Sons, New York, N.Y., pp. 22.0.1-22.3.26; Huang, et al. (2000) Cancer Res 60:6868-6874). Screening of cells and tissues is described (Ausubel, et al. (2001) Curr. Protocols Mol. Biol., Vol. 4, John Wiley and Sons, New York, N.Y., pp. 25.0.1-25B.2.20 and Ausubel, et al. (2001) Curr. Protocols Mol. Biol., Vol. 3, John Wiley and Sons, New York, N.Y., pp. 14.0.1-14.14.8).

Variations and hybrids of the above techniques may be used, i.e., antibodies bearing covalently linked DNA may be used as probes, where bound antibody is detected by the PCR method (Sims, et al. (2000) Analyt. Biochem. 281:230-232). The above techniques can also be used for screening of therapeutic agents that modulate the expression, processing, secretion, and binding functions of C1QSF3.

V. Protein Purification.

It is contemplated to purify the polypeptide diagnostics or therapeutics of the invention, such as antigens, antibodies, and antibody fragments, by methods that are established in the art.

Initial stages of purification may include homogenization of cells, i.e., by a sonicator, French press, or blender, and selective precipitation by fractionation with ammonium sulfate, polyethylene glycol, or solvents (Dennison and Lovrien (1997) Protein Expression Purif. 11:149-161). It is recognized that protein integrity during purification may be enhanced by including solvents such as glycerol or sucrose, reductants such as dithiothreitol (DTT), anti-protease “cocktails,” albumin supplements, reduced temperatures, and by genetic engineering of the protein itself (Murby, et al. (1996) Protein Expression Purif. 7:129-136). Contaminating proteases, or auto-proteolytic action, can cleave peptide bonds at all stages of purity of a protein, where any resulting change in function depends, e.g., on the position of cleavage and on the protein target.

Polypeptides may be purified by ion exchange chromatography, such as diethylaminoethylcellulose, phosphocellulose, and carboxymethylcellulose, or by molecule sieve chromatography, as with Sephadex® and Sephacryl® gel filtration media (Pharmacia catalogue (2001) Amersham Pharnacia Biotech, Inc., Piscataway, N.J.). Polypeptides may be purified by affinity chromatography, as with immobilized lectins, immobilized antibodies, immobilized ligands or substrates, and immobilized hydrophobic arms. Glycoprotein biochemistry and purification by lectins is described (Ausubel, et al. (2001) Curr. Protocols Mol. Biol., Vol. 3, John Wiley and Sons, New York, N.Y., pp. 17.0.1-17.23.8). Polypeptides may be engineered to contain a tag specially formulated to facilitate purification, expressed in a host cell, and then purified. Such tags include an oligohistidine tag (Rajan, et al. (1998) Protein Expression Purif. 13:67-72), glutathione S-transferase, streptavidin, or protein A, for example (Pharmacia catalogue (2001) Amersham Pharmacia Biotech, Inc., pp. 543-567, 605-654). The immobilized ligands for these tags are nickel, glutathione, biotin, and Fc fragment, respectively. Machine-based methods of protein purification include preparative isoelectric focusing, preparative polyacrylamide gel electrophoresis, two dimensional electrophoretic gels, and systems operating under elevated pressure, such as high pressure liquid chromatography (HPLC) (Gooding and Regnier (2002) HPLC of Biological Molecules, 2^{nd} ed., Marcel Dekker, NY; Cunico, Gooding, and Wehr (1998) Basic HPLC and CE of Biomolecules, Bay Biological Laboratory, Inc. Hercules, Calif.). Instruments such as the BioCAD® (Applied Biosystems, Foster City, Calif.) allow the automated generation of pH gradients, salt and buffer gradients, and flow rates during protein purification, thus facilitating the determination of a workable purification protocol.

Membrane-bound proteins may require a different type of detergent at the various steps in a purification procedure. Different types of detergent may be required for initial solubilization, for maintenance of solubility during chromatographic purification, and during assay of biological or antigenic activity. For initial solubilization, ionic detergents such as sodium cholate, or non-ionic detergents such as 3-[(3-cholamidopropyl)dimethylammonio]-1-propanesulfonate (CHAPS) or Triton X-100®, may be suitable. However, zwitterionic detergents, such as CHAPS, or non-ionic detergents, such as Triton X-100, may be required during fractionation on an ion exchange column. Where the protein is to be maintained in a non-denatured, biologically active state, the detergent may be Triton X-100, Tween 20, Brij 58, CHAPS, cholate, deoxycholate, or other detergents or stabilizers, as described, see, e.g., Weiner and Kotkoskie (2000) Excipient Toxicity and Safety, Marcel Dekker, Inc., New York, N.Y. and Sigma catalogue (2002) Sigma-Aldrich, Co., St. Louis, Mo.

VI. Antibodies.

Antibodies can be raised to various embodiments of C1QSF3, including species, polymorphic, allelic, and mutational variants, and fragments thereof, both in their naturally occurring forms and in their recombinant forms. Additionally, antibodies can be raised to C1QSF3 in either their active forms or in their inactive forms, including native or denatured versions. Anti-idiotypic antibodies are also contemplated.

Antibodies, including humanized antibodies, monoclonal antibodies, and binding fragments, such as Fab, F(ab)₂, and Fv fragments are contemplated. Antibodies may be agonistic or antagonistic, by binding to a protein, e.g., a ligand or a receptor, and inhibiting or stimulating the binding of that protein to its receptor. Antibodies that simultaneously bind to a ligand and receptor are contemplated. Monoclonal antibodies will usually bind with at least a K_{D} of about 1 mM, more usually at least about 300 μM, typically at least about 100 μM, more typically at least about 30 μM, preferably at least about 10 μM, and more preferably at least about 3 μM or better.

While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments produced by the modification of whole antibodies, or those synthesized de novo using recombinant DNA methodologies, such as recombinant IgG antibodies (U.S. Pat. No. 4,816,567 issued to Cabilly, et al; U.S. Pat. No. 4,642,334 issued to Moore, et al.; Queen, et al. (1989) Proc. Natl Acad. Sci. USA 86:10029-10033), single chain antibodies, or antibodies acquired by phage display, and monoclonal antibodies made by the hybridoma method (Kohler, et al. (1975) Nature 256:495-497).

For preparation of monoclonal or polyclonal antibodies, any technique known in the art can be used (see, e.g., Kozbor, et al. (1983) Immunology Today 4:72; Cole, et al. (1985) in Monoclonal Antibodies and Cancer Therapy, Alan R. Liss, Inc., New York, N.Y., pp. 77-96). Monoclonal antibodies are generally derived from non-human sources, rather than from human sources (Harlow and Lane (1988) Antibodies A Laboratory Manual, Cold Spring Harbor Laboratory Press, Cold Spring Harbor, N.Y., pp. 139-243).

The use of non-human sources can limit the therapeutic efficiency of a monoclonal antibody. Antibodies derived from non-human sources can provoke an immune response, weak recruitment of effector function, and rapid clearance from the bloodstream (Baca, et al. (1997) J. Biol. Chem. 272:10678-10684). For these reasons, it may be desired to prepare therapeutic antibodies by humanization (Carpenter, et al. (2000) J. Immunol. 165:6205; He, et al. (1998) J. Immunol. 160:1029; Tang, et al. (1999) J. Biol. Chem. 274:27371-27378). A humanized antibody contains the amino acid sequences from six complementarity determining regions (CDRs) of the parent mouse antibody, which are grafted on a human antibody framework. To achieve optimal binding, the humanized antibody may need fine-tuning, by changing certain framework amino acids, usually involved in supporting the conformation of the CDRs, back to the corresponding amino acid found in the parent mouse antibody.

An alternative to humanization is to use human antibody libraries displayed on phage (Vaughan, et al. (1996) Nat. Biotechnol. 14:309-314; Barbas (1995) Nature Med. 1:837-839; de Haard, et al. (1999) J. Biol. Chem. 274:18218-18230; McCafferty et al. (1990) Nature 348:552-554; Clackson et al. (1991) Nature 352:624-628; Marks et al. (1991) J. Mol. Biol. 222:581-597), or human antibody libraries contained in transgenic mice (Mendez, et al. (1997) Nature Genet. 15:146-156). The phage display technique can be used for screening for and selecting antibodies with high binding affinity (Hoogenboom and Chames (2000) Immunol. Today 21:371-377; Barbas, et al. (2001) Phage Display: A Laboratory Manual, Cold Spring Harbor Laboratory Press, Cold Spring Harbor, N.Y.; Kay, et al. (1996) Phage Display of Peptides and Proteins: A Laboratory Manual, Academic Press, San Diego, Calif.). Use of the phage display method can provide a DNA sequence that provides a tight binding monovalent antibody, as displayed on the surface of filamentous phage. With this DNA sequence in hand, the researcher can build a tight binding humanized bivalent antibody. A phage display library may comprise single chain antibodies where heavy and light chain variable regions are fused by a linker in a single gene, or it may comprise co-expressed heavy and light chains (de Bruin, et al. (1999) Nat. Biotechnol. 17:397-399).

Single chain antibodies are described, e.g., in Malecki, et al. (2002) Proc. Natl. Acad. Sci. USA 99:213-218 and U.S. Pat. No. 4,946,778 issued to Ladner, et al., while single domain antibodies are described by Conrath, et al. (2001) J. Biol. Chem. 276:7346-7350 and Desmyter, et al. (2001) J. Biol. Chem. 276:26285-26290). Bispecific antibodies are described. See, e.g., U.S. Pat. Nos. 5,932,448 issued to Tso, et al., 5,532,210 issued to Paulus, and 6,129,914 issued to Weiner, et al. Bispecific antibodies may be synthesized using leucine zipper technology (Kostelney, et al. (1992) J. Immunol. 148:1547-1553; U.S. Pat. No. 6,133,426 issued to Gonzalez, et al.).

Purification of antigen is not necessary for the generation of antibodies. Immunization can be performed by DNA vector immunization. See, e.g., Wang, et al. (1997) Virology 228:278-284. Alternatively, animals can be immunized with cells bearing the antigen of interest. Splenocytes can then be isolated from the immunized animals, and the splenocytes can fused with a myeloma cell line to produce a hybridoma (Meyaard, et al. (1997) Immunity 7:283-290; Wright, et al. (2000) Immunity 13:233-242; Preston, et al. (1997) Eur. J. Immunol. 27:1911-1918). Resultant hybridomas can be screened for production of the desired antibody by means of functional assays or biological assays, that is, assays not dependent on possession of the purified antigen. Immunization with cells may prove superior for antibody generation than immunization with purified antigen (Kaithamana, et al. (1999) J. Immunol. 163:5157-5164).

Antigen fragments may be joined to other materials, such as fused or covalently joined polypeptides, to be used as immunogens. An antigen and its fragments may be fused or covalently linked to a variety of immunogens, such as keyhole limpet hemocyanin, bovine serum albumin, or ovalbumin (Coligan, et al. (1994) Current Protocols in Immunol., Vol. 2, Unit 9.3-9.4, John Wiley and Sons, New York, N.Y.). Peptides of suitable antigenicity can be selected from the polypeptide target, using an algorithm, such as those of Parker, et al. (1986) Biochemistry 25:5425-5432; Jameson and Wolf (1988) Cabios 4:181-186; or Hopp and Woods (1983) Mol. Immunol. 20:483-489.

The signal sequence or leader sequence can be predicted (Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

Antibody to antigen binding properties can be measured, e.g., by surface plasmon resonance (Karlsson, et al. (1991) J. Immunol. Methods 145:229-240; Neri, et al. (1997) Nat. Biotechnol. 15:1271-1275; Jonsson, et al. (1991) Biotechniques 11:620-627) orby competition ELISA (Friguet, et al. (1985) J. Immunol. Methods 77:305-319; Hubble (1997) Immunol. Today 18:305-306).

A variety of approaches are used to make therapeutic antibodies, or fragments thereof. See, e.g., (Yip and Ward (2002) Cancer Immunol. Immunother. 50:569-587; U.S. Pat. No. 5,772,997 issued to Hudziak, et al.; Lin and Castro (1998) Curr. Opinion Chem. Biol. 2:453-457; Rader, et al. (1998) Proc. Natl. Acad. Sci. USA 95:8910-8915; Targan, et al. (1997) New Engl. J. Med. 337:1029; Joosten, et al. (1996) Arthritis and Rheumatism 39:797).

Therapeutic antibodies occurring as conjugated antibodies or fusion protein antibodies are described. Antibodies may be conjugated to toxins (van Oosterhout, et al. (2001) Int. J. Pharm. 221:175-186; Marsh and Klinman (1990) J. Immunol. 144:1046-1051; Kreitman (2001) Curr. Pharm. Biotechnol. 2:313-325; Dinndorf, et al. (2001) J. Immunother. 24:511-516), small drug molecules (Wahl, et al. (2001) Int. J. Cancer 93:540-600; Garber (2000) J. Nat. Cancer Instit. 92:1462-1464; Everts, et al. (2002) J. Immunol. 168:883-889), enzymes for generating an active drug from a pro-drug (Chen, et al. (2001) Int. J. Cancer 94:850-858), to liposomes (Shaik, et al. (2001) J. Control. Release 76:285-295; Park, et al. (2001) J. Control. Release 74:95-113), polyethylene glycol (PEG) (Solorzano, et al. (1998) J. Appl. Physiol. 84:1119-1130; Rosenberg, et al. (2001) J. Appl. Physiol. 91:2213-2223; Bendele, et al. (2000) Arthritis Rheum. 43:2648-2659; Trakas and Tzartos (2001) J. Neurochem. 120:42-49; Chapman, et al. (1999) Nat. Biotechnol. 17:780-783), and recognition tags (Gaidamakova, et al. (2001) J. Control. Release 74:341-347).

Conjugated antibodies are useful for diagnostic or kit purposes, and include antibodies coupled to dyes, such as fluorescein or phycoerythrin, radioactive atoms, such as iodine-125, enzymes, such as horse radish peroxidase (Le Doussal, et al. (1991) J. Immunol. 146:169-175; Gibellini, et al. (1998) 160:3891-3898; Hsing and Bishop (1999) J. Immunol. 162:2804-2811), colloidal gold (Everts, et al. (2002) J. Immunol. 168:883-889), or various other moieties (Harlow and Lane (1988) supra). The binding of diagnostic antibodies to cells can be measured by immunochemistry or by flow cytometry (Everts, et al. (2002) J. Immunol. 168:883-889). See also, Chan (1987) Immunology: A Practical Guide, Academic Press, Orlando, Fla.; Price and Newman (1991) Principles and Practice of Immunoassay, Stockton Press, N.Y.; and Ngo (1988) Nonisotopic Immunoassay, Plenum Press, N.Y.

The antibodies of this invention can also be used for affinity chromatography in isolating the antibody's target antigen or related proteins. Columns can be prepared where the antibodies are linked to a solid support. See, e.g., Wilchek, et al. (1984) Meth. Enzymol. 104:3-55.

Antibodies to C1QSF3 having substantially the same nucleic acid and amino acid sequence as those recited herein, but possessing substitutions that do not substantially affect the functional aspects of the nucleic acid or amino acid sequence, are within the definition of the contemplated invention. Variants with truncations or deletions of regions which do not change the biological functions of these nucleic acids and polypeptides are also within the definition of the contemplated invention.

VII. Therapeutic Compositions.

Formulations of antibodies, binding composition, polypeptides, or small molecule therapeutics are prepared for storage by mixing with physiologically acceptable carriers, excipients, or stabilizers in the form of, e.g., lyophilized powders, slurries, aqueous solutions. See, e.g., Hardman, et al. (2001) Goodman and Gilman's the Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; and Lieberman, et al. (eds.) (1990) Pharmaceutical Dosage Forms: Disperse Systems Dekker, N.Y.

Carriers, excipients, detergents, surfactants, and stabilizers are described, see, e.g., Weiner and Kotkoskie (2000) Excipient Toxicity and Safety, Marcel Dekker, Inc., New York, N.Y.; Sigma Catalogue (2002) Sigma-Aldrich, Co., St. Louis, Mo.; U.S. Pat. No. 6,096,728 issued to Collins, et al.; U.S. Pat. No. 6,342,220 issued to Adams, et al.; U.S. Pat. No. 5,440,021 issued to Chuntharapai, et al.

The antibody, binding composition, polypeptide, or small molecule to be used for in vivo administration must be sterile. This is readily accomplished by filtration through sterile filtration membranes, prior to or following lyophilization and reconstitution. The antibody ordinarily will be stored in lyophilized form or in solution.

Therapeutic compositions comprising an antibody or small molecule can be administered, e.g., by systemic, intraperitoneal, intramuscular, and intratumor routes. Sustained-release preparations (Sidman et al. (1983) Biopolymers, 22:547-556; Langer et al. (1981) J. Biomed. Mater. Res. 15:167-277; Langer (1982) Chem. Tech. 12:98-105; U.S. Pat. Nos. 6,387,404; 6,375,972), liposomes (Epstein et al. (1985) Proc. Natl. Acad. Sci. USA, 82:3688-3692; Hwang et al. (1980) Proc. Natl. Acad. Sci. USA, 77:4030-4034; U.S. Pat. Nos. 6,387,397; 6,379,699; 6,372,720; 6,348,214; 6,290,987; 6,372,259; 6,335,035; 6,328,979; 6,312,728), or aerosols may be used to supply the therapeutic composition. Adenovirus and other vectors are also contemplated as a delivery agent for the contemplated invention (See, e.g., U.S. Pat. Nos. 6,387,368; 6,379,943; 6,297,220; 6,281,010; and 6,245,966).

An “effective amount” of antibody or other therapeutic to be employed will depend, for example, upon the therapeutic objectives, the route of administration, the type of antibody employed, and the condition of the patient. Accordingly, it will be necessary for the therapist to titer the dosage and modify the route of administration as required to obtain the optimal therapeutic effect. Typically, the clinician will administer the antibody until a dosage is reached that achieves the desired effect. The progress of this therapy is easily monitored by conventional assays.

In the treatment and prevention of an inflammatory disorder the therapeutic composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the antibody, the particular type of antibody, the method of administration, the scheduling of administration, and other factors known to medical practitioners. The “therapeutically effective amount” of antibody to be administered will be governed by such considerations, and is the minimum amount necessary to prevent, ameliorate, or treat the proliferative disorder. Such amount is preferably below the amount that is toxic to the host.

As a general proposition, the initial pharmaceutically effective amount of the antibody administered parenterally will be in the range of about 0.1 to 50 mg/kg of patient body weight per day, with the typical initial range of antibody used being 0.3 to 20 mg/kg/day, more preferably 0.3 to 15 mg/kg/day. The desired dosage can be delivered by a single bolus administration, by multiple bolus administrations, or by continuous infusion administration of antibody, depending on the pattern of pharmacokinetic decay that the practitioner wishes to achieve.

The therapeutic may be formulated with one or more agents currently used to prevent or treat an immune condition, autoimmune condition, or inflammatory condition.

VIII. Kits.

This invention also contemplates use of C1QSF3 proteins and fragments thereof, nucleic acids, and fragments thereof, such as primers and probes, in a diagnostic kit. The invention also contemplates use of binding composition, including antibodies or antibody fragments, for the detection or quantitation of C1QSF3 proteins, C1QSF3, and breakdown products. Typically, the kit will have a compartment containing either a C1QSF3 polypeptide, nucleic acid coding for part or all of a C1QSF3, or a reagent that recognizes the polypeptide or polypeptide fragment, e.g., an antibody or receptor fragment. The invention also contemplates nucleic acids, nucleic acid fragments, or nucleic acid probes or primers.

For example, a kit for determining the binding of a test compound, e.g., acquired from a biological sample or from a chemical library, would typically comprise a control compound, a labeled compound, and a means for separating free labeled compound from bound labeled compound. The means for separating free labeled from bound labeled compound may be a solid phase matrix containing an antibody that binds the test compound and the labeled compound. Diagnostic assays can be used with biological matrices such as live cells, lysates, fixed cells, cell cultures, bodily fluids, forensic samples. Various commercial assays exist, such as radioimmunoassays (RIA), enzyme-linked immunoassay (ELISA), substrate-labeled fluorescent immunoassay, and the like.

Numerous methods exist for the separation of bound ligand from free ligand, or bound test compound from free test compound. Methods of immobilizing ligands or test compounds include, without limitation, direct adhesion to plastic, use of a capture antibody, chemical coupling, biotin-avidin couplings, and biotin-streptavidin couplings. Another approach for the separation of bound from free test compounds is to use an organic solvent to lake advantage of differences in relative solubility, or a precipitant such as polyethylene glycol (PEG), ammonium sulfate, or other salt.

IX. Uses.

The present invention provides reagents which will find use in diagnostic and therapeutic applications, e.g., relating to the modulation of the activity, behavior, and development of cells, including monocytes, mast cells, dendritic cells, macrophages, lymphocytes, NK cells, hematopoietic precursors, neutrophils, and epithelial cells. The reagents will also find use in applications relating to the development or physiology of various tissues and organs, e.g., epithelial tissues, endothelial tissues, lungs, heart, skin, small and large intestines, joints, thyroid, spinal cord, and the central nervous system.

The present invention provides C1QSF3, isolated fragments of C1QSF3, analogs or muteins thereof, and binding compositions specific for C1QSF3, or a derivative or fragment thereof, for use in the treatment of conditions associated with abnormal physiology or development, e.g., relating to the heart, lungs, gut, joints, and cells of the immune system. The C1QSF3, binding composition thereto, and fragments and analogues thereof, are expected to be of use for the treatment of pathological conditions, e.g., inflammation, infection, abnormal proliferation, cancer, metastasis, or pathological cell adhesion. The invention is expected to be of use for the diagnosis or treatment of psoriasis, Hashimoto's thyroiditis, asthma, or inflammatory bowel disease, e.g., Crohn's disease.

For example, a disease condition associated with abnormal expression or abnormal signaling by C1QSF3 should be a likely target for an agonist or antagonist of an antigen of C1QSF3. Thus, C1QSF3 will likely modulate interactions of cells of the lung, skin, or joint with other cell types, e.g., cells which possess a receptor therefor. These interactions would lead, in particular contexts, to modulation of cell growth, cytokine synthesis by those or other cells, or development of particular effector cells.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

### EXAMPLES

I. General Methods.

Some of the standard methods are described or referenced, e.g., in Maniatis, et al. (1982) Molecular Cloning, A Laboratory Manual, Cold Spring Harbor Laboratory, Cold Spring Harbor Press; Sambrook and Russell (2001) Molecular Cloning, 3^{rd.} ed., Cold Spring Harbor Laboratory Press, Cold Spring Harbor, N.Y.; Wu (1993) Recombinant DNA, Vol. 217, Academic Press, San Diego, Calif.; Innis, et al. (eds.) (1990) PCR Protocols: A Guide to Methods and Applications, Academic Press, N.Y. Standard methods are also found in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4). Methods for producing transgenic animals are described, e.g., in Jackson and Abbott (eds.) (2000) Mouse Genetics and Transgenics, Oxford Univ. Press, Oxford, UK; Hofker, et al. (eds.) (2002) Transgenic Mouse Methods and Protocols, Humana Press, Clifton, N.J.

Methods for protein purification such as ammonium sulfate fractionation, column chromatography, electrophoresis, isoelectric focusing, centrifugation, and crystallization, are described (Coligan, et al. (2000) Current Protocols in Protein Science, Vol. 1, John Wiley and Sons, Inc., New York). Chemical analysis, chemical modification, post-translational modification, and glycosylation of proteins is described. See, e.g., Coligan, et al. (2000) Current Protocols in Protein Science, Vol. 2, John Wiley and Sons, Inc., New York; Walker (ed) (2002) Protein Protocols Handbook, Humana Press, Towota, N.J.; Lundblad (1995) Techniques in Protein Modification, CRC Press, Boca Raton, Fla. The production, purification, and fragmentation of polyclonal and monoclonal antibodies is described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.); Harlow and Lane (1988) supra).

Standard techniques for characterizing ligand/receptor interactions are described (Coligan, et al. (2001) Current Protcols in Immunology, Vol. 4, John Wiley and Sons, Inc., New York).

Cell culture techniques are described in Doyle, et al. (eds.) (1994) Cell and Tissue Culture: Laboratory Procedures, John Wiley and Sons, NY. FACS analysis is described in Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

Reagents for fluorescent labeling of proteins, nucleic acids, lipids, carbohydrates, and cells are from Molecular Probes, Inc. (Eugene, Oreg.). These reagents include those that modify thiols, amines, hydroxylated molecules, fluorescent biotin derivatives, and photoreactive reagents.

II. Identification of Human C1QSF.

A structure-guided alignment for the TNF family was constructed and an HMM (Hidden Markov model) was built from the HMM. The HMM was used to in an hframe search versus the Schering human contigs. Hframe is an algorithm that allows you to search a nucleotide database with a protein HMM. The sequences from the hframe run were compared by blast to a set of known TNF and C1Q sequences. Sequences that were not known, i.e., sequences that shared less than 95% homology to a known protein, were investigated further. C1QSF1-5 (human) were identified from the output of this search. Mouse sequences where identified by Blasting the human sequences vs. the mouse EST database. C1QSF6-11 human and mouse sequences were identified by blasting C1QSF1-5 against human and mouse EST and protein databases. Clones were obtained for these genes, and their sequences were confirmed.

III. Cellular Expression of Mammalian C1QSF3.

Tissue and cell distribution of C1QSF expression was determined by TaqMan® analysis (PE Applied Biosystems, Foster City, Calif.). Expression from human and murine cells and tissues is shown (Table 2).

TABLE 2
Expression ot C1QSF3 in cells and tissues.
(−) Means little or no detectable expression.

| | |
|---|---|
| Skin control (human) | + |
| Skin psoriasis (human) | +++ |
| Colon control (human) | + |
| Colon Crohn's (human) | +++ |
| Thyroid control (human) | ++ |
| Thyroid Hashimoto's thyroiditis (human) | +++++ |
| Lung control (human) | ++++++ |
| Lung idiopathic pulmonary fibrosis (human) | ++++++++ |
| Lung hypersensitivity pneumonitis (human) | +++++++ |
| Heart fetal (human) | ++++++++ |
| Small intestines fetal (human) | ++ |
| Hematopoietic precursor, activated (human) | ++++++ |
| Premonocyte resting (human) | ++ |
| Monocyte activated with lipopolysaccharide (LPS) (human) | +++ |
| T cell TH1 resting (human) | − |
| T cell TH2 resting (human) | − |
| Epithelial cell bronchial (human) | − |
| Spleen C57BL/6, untreated (murine) | − |
| Spleem C57BL/6, TNFα knockout, untreated (murine) | +++++ |
| Lung BALB/c untreated (murine) | + |
| Lung asthma model OVA challenged (murine) | ++++ |
| Lung tolerized OVA challenged (murine) | + |
| Lung infected Nippostrongulus (murine) | + |

IV. Preparation of Antibodies that Bind C1QSF3.

Analysis of human C1QSF3 (SEQ ID NO:10) for antigenicity using a Welling plot (Vector NTI® Suite V.7, InforMax®, Bethesda, Md.) revealed a number of regions of increased antigenicity. These regions approximately include amino acid residues 100-240, 280-295, 590-610, 770-810, and 970-120.

Many modifications and variations of this invention, as will be apparent to one of ordinary skill in the art can be made to adapt to a particular situation, material, composition of matter, process, process step or steps, to preserve the objective, spirit, and scope of the invention. All such modifications are intended to be within the scope of the claims appended hereto without departing from the spirit and scope of the invention. The specific embodiments described herein are offered by way of example only, and the invention is to be limited by the terms of the appended claims, along with the full scope of the equivalents to which such claims are entitled; and the invention is not to be limited by the specific embodiments that have been presented herein by way of example.

### DESCRIPTION OF SEQUENCE IDENTIFICATION NUMBERS

SEQ ID NO: 1 is human C1QSF1 nucleic acid sequence

SEQ ID NO: 2 is human C1QSF1 polypeptide sequence

SEQ ID NO: 3 is murine C1QSF1 nucleic acid sequence

SEQ ID NO: 4 is murine C1QSF1 polypeptide sequence

SEQ ID NO: 5 is human C1QSF2/5 nucleic acid sequence

SEQ ID NO: 6 is human C1QSF2/5 polypeptide sequence

SEQ ID NO: 7 is murine C1QSF2/5 nucleic acid sequence

SEQ ID NO: 8 is murine C1QSF 2/5 polypeptide sequence

SEQ ID NO: 9 is human C1QSF3 nucleic acid sequence

SEQ ID NO: 10 is human C1QSF3 polypeptide sequence

SEQ ID NO: 11 is murine C1QSF3 nucleic acid sequence

SEQ ID NO: 12 is murine C1QSF3 polypeptide sequence

SEQ ID NO: 13 is human C1QSF4 nucleic acid sequence

SEQ ID NO: 14 is human C1QSF4 polypeptide sequence

SEQ ID NO: 15 is murine C1QSF4 nucleic acid sequence

SEQ ID NO: 16 is murine C1QSF4 polypeptide sequence

SEQ ID NO: 17 is human C1QSF6 nucleic acid sequence

SEQ ID NO: 18 is human C1QSF6 polypeptide sequence

SEQ ID NO: 19 is murine C1QSF6 nucleic acid sequence

SEQ ID NO: 20 is murine C1QSF6 polypeptide sequence

SEQ ID NO: 21 is human C1QSF7 nucleic acid sequence

SEQ ID NO: 22 is human C1QSF7 polypeptide sequence

SEQ ID NO: 23 is murine C1QSF7 nucleic acid sequence

SEQ ID NO: 24 is murine C1QSF7 polypeptide sequence

SEQ ID NO: 25 is human C1QSF8 nucleic acid sequence

SEQ ID NO: 26 is human C1QSF8 polypeptide sequence

SEQ ID NO: 27 is murine C1QSF8 nucleic acid sequence

SEQ ID NO: 28 is murine C1QSF8 polypeptide sequence

SEQ ID NO: 29 is human C1QSF9 nucleic acid sequence

SEQ ID NO: 30 is human C1QSF9 polypeptide sequence

SEQ ID NO: 31 is murine C1QSF9 nucleic acid sequence

SEQ ID NO: 32 is murine C1QSF9 polypeptide sequence

SEQ ID NO: 33 is human C1QSF11 nucleic acid sequence

SEQ ID NO: 34 is human C1QSF11 polypeptide sequence

SEQ ID NO: 35 is murine C1QSF11 nucleic acid sequence

SEQ ID NO: 36 is murine C1QSF11 polypeptide sequence

What is claimed is:

1. An isolated nucleic acid encoding a polypeptide comprising SEQ ID NO:10 or 12, or an antigenic fragment thereof.

2. An isolated nucleic acid which hybridizes to the nucleic acid of claim 1, under stringent conditions.

3. The nucleic acid of claim 1 comprising SEQ ID NO:9 or 11.

4. An expression or replicating vector comprising the nucleic acid of claim 1.

5. A host cell comprising the expression or replicating vector of claim 4.

6. An isolated polypeptide comprising SEQ ID NO:10 or 12, or an antigenic fragment thereof.

7. The polypeptide of claim 6, further comprising a fusion polypeptide or peptide.

8. A binding composition which specifically binds to the polypeptide of claim 6.

9. The binding composition of claim 8, wherein the binding composition comprises an antigen binding site of an antibody.

10. The binding composition of claim 9, wherein the binding composition is a polyclonal or monoclonal antibody.

11. The binding composition of claim 9, wherein the binding composition is a humanized antibody.

12. The binding composition of claim 9, wherein the binding composition is an Fab fragment, an F(ab′)₂ fragment, or an Fv fragment.

13. The binding composition of claim 9, wherein the binding composition is detectably labeled.

14. The binding composition of claim 8 in conjunction with an acceptable carrier.

15. A kit comprising:
a) the substantially pure polypeptide of claim 6;
b) a binding composition which specifically binds the polypeptide; or
c) a nucleic acid encoding the polypeptide.

16. A method of producing a polypeptide comprising SEQ ID NO:10 or 12, or an antigenic fragment thereof, comprising:
a) culturing the host cell of claim 5 under conditions suitable for expression of the polypeptide; and
b) isolating or purifying the polypeptide.

17. A method of modulating the activity of a cell comprising contacting the cell with the binding composition of claim 8.

18. A method of treating a subject suffering from an inflammatory condition comprising administering an effective amount of an agonist or antagonist of the polypeptide of claim 6.

19. The method of claim 18, wherein the agonist or antagonist is a binding composition which specifically binds to a polypeptide comprising SEQ ID NO:10 or 12, or an antigenic fragment thereof.
